(19) United States
(12) Patent Application Publication
(10) Pub. No.: US 2026/0069524 A1
(43) Pub. Date: Mar. 12, 2026

(54) COSMETIC AND PERSONAL CARE COMPOSITIONS COMPRISING RECOMBINANT SILK

(71) Applicant: BOLT THREADS , INC., Emeryville, CA (US)

(72) Inventor: Lindsay Wray, Emeryville, CA (US)

(21) Appl. No.: 18/706,281

(22) PCT Filed: Nov. 2, 2022
(86) PCT No.: PCT/US2022/079160

§ 371 Date: Apr. 30, 2024

Related U.S. Application Data
(60) Provisional application No. 63/274,837, filed on Nov. 2, 2021.
(60) Provisional application No. 63/302,793, filed on Jan. 25, 2022.

(51) Int. Cl. A61K 8/64; A61K 8/04; A61K 8/34; A61K 8/365; A61K 8/368; A61K 8/41; A61K 8/49; A61K 8/81; A61Q 1/02; A61Q 5/00; A61Q 5/02; A61Q 5/06; A61Q 17/04; A61Q 19/00

(52) U.S. Cl. CPC A61K 8/64; A61K 8/042; A61K 8/345; A61K 8/365; A61K 8/368; A61K 8/416; A61K 8/498; A61K 8/8152; A61Q 1/02; A61Q 5/00; A61Q 5/02; A61Q 5/06; A61Q 17/04; A61Q 19/00; A61K 2800/34; A61K 2800/43; A61K 2800/51; A61K 2800/524

(57) ABSTRACT

Disclosed herein is a composition comprising recombinant silk polypeptide and use thereof as silicone (both “fluid” and “elastomer”) replacement in beauty and personal care formulations for influencing skin and hair properties and can include benefits to the formulations themselves, such as increasing formulation viscosity.

### FIELD OF THE INVENTION

The present invention relates to a composition comprising recombinant silk polypeptide and use thereof as silicone (both “fluid” and “elastomer”) replacements in beauty and personal care formulations for influencing skin and hair properties and can include benefits to the formulations themselves, such as increasing formulation viscosity. For skin, these properties can include long-lasting wear, silky soft feel, matte finish, enhanced pigment delivery, spreadability, quick absorption, wrinkle blurring effect, and UV and pollution defense. For hair, these properties can include long-lasting wear, shine, non-greasiness, frizz control, adding thickness to the hair, styling retention, resistance to heat, and UV and pollution defense.

### BACKGROUND

Silicone polymers are a broad chemical family with the commonality of alternating silicon and oxygen atoms making up the backbone of the polymer. Generally speaking, “liquid” silicone polymers or “silicone fluids” refers to high molecular weight silicone polymers that may or may not contain functional groups. These polymers can also be referred to as “dimethicone”. Silicone elastomers refer to the types of silicones where the linear silicone polymer has been crosslinked to form a gel-like network.

Silicone fluids and silicone elastomers are widely used in the beauty and personal care industry. For example silicone elastomers are known for leaving the skin feeling soft and velvety as well as imparting a matte finish to the skin. Some common applications of silicones are BB creams, anti-aging wrinkle reducers, primers, liquid foundations, mousse foundations, gelled eye shadows and many more.

While fluid silicones and silicone elastomers provide benefits to skin, hair, and personal care applications, silicones are harder to wash off and can get stuck in pores. This is because silicones are hydrophobic and they repel water. For this reason, silicone-based products don't rinse away easily. Also silicones are not eco-friendly-they have an extremely slow biodegradation rate in the environment and as such they can bioaccumulate. Once they are rinsed down the drain, they contribute to the buildup of sludge pollution in oceans and waterways and may not break down for decades or even hundreds of years (Horii Y., Kannan K. (2019) Main Uses and Environmental Emissions of Volatile Methylsiloxanes. In: Homem V., Ratola N. (eds) Volatile Methylsiloxanes in the Environment. The Handbook of Environmental Chemistry, vol 89. Springer, Cham. https://doi.org/10.1007/698_2019_375).

To date, there is an unmet need for silicone elastomer alternative ingredients that meet the same performance level as silicone elastomers while also being biobased and biodegradable. Many ingredients aspect to make a “greener” silicone elastomer by replacing the cyclosiloxane diluent (which usually accompanies silicone elastomers) with a biobased alternative. While this is a step in the right direction, it still falls very short in solving the problem at hand, which is that the silicone elastomer is still not biodegradable.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1A illustrates a top-down view of the SPF formulations.

FIG. 1B shows a spider chart plotting data from a blind test comparison of SPF formulations.

FIG. 1C shows ingredients used in all SPF formulations (not including recombinant silk polypeptide and silicones).

FIG. 1D illustrates elastic modulus, viscous modulus and phase angle vs. frequency for SPF base formulation, SPF formulation with 1.5% recombinant silk polypeptide, and SPF formulation with 2% silicones.

FIG. 1E shows light microscopy images of SPF base and SPF base formulated with 1% b-silk protein (i.e., an 18B silk; a recombinant silk comprising SEQ ID NO: 2878) or 5% silicone elastomer ingredient. A reference image of 1% recombinant silk polypeptide powder suspended in water is also shown.

FIG. 2A illustrates a top-down view of 3-in-1 cream eye/cheek/lip formulation (i.e., color cosmetic) with 1% recombinant silk polypeptide or 10% silicone elastomer.

FIG. 2B illustrates a flow chart for evaluation of the color cosmetic for pigment delivery and substantiation to skin.

FIG. 2C shows representative images of color cosmetic application and wipe-off for a color cosmetic with i) 1% recombinant silk polypeptide, ii) 5% silicone elastomer, and iii) 10% silicone elastomer.

FIG. 2D shows ingredients used in all color cosmetic formulations (not including recombinant silk polypeptide and silicones).

FIG. 2E shows light microscopy images of color cosmetic base and the base formulated with 1% recombinant silk polypeptide or 5% silicone elastomer ingredient.

FIG. 2F shows a chart of elastic modulus, viscous modulus and phase angle vs. frequency for color cosmetic base, color cosmetic with 1% recombinant silk polypeptide, and color cosmetic with 5% silicone elastomer.

FIG. 3A shows light microscopy images of hair serum formulations with and without 1% recombinant silk polypeptide and 5% silicone elastomer. A reference image of 1% recombinant silk polypeptide powder suspended in water is also shown.

FIG. 3B shows SEM images of yak hair at 150× and 800× magnification that has i) not been treated, ii) treated with serum base, iii) treated with serum base with 1% recombinant silk protein, and iv) treated with serum base with 5% elastomer.

FIG. 3C shows a chart of elastic modulus, viscous modulus and phase angle vs. frequency (bottom row) or vs. shear strain (top row) for color hair serum base, hair serum base with 1% recombinant silk protein, and hair serum base with 5% silicone elastomer.

FIG. 3D is a table of ingredients used in the hair serum base formulation (not including the recombinant silk polypeptide and silicone elastomers).

FIG. 4A shows a comparison of the G′ and G″ of an industry standard silicone elastomer gel (dry solids of 30%) and 12% recombinant silk polypeptide on a chart of elastic modulus, viscous modulus and phase angle vs. frequency (top row) or viscosity vs. shear rate (bottom row).

FIG. 4B shows SEM images of the neat recombinant silk polypeptide and silicone elastomer dispersed onto yak hair at 150× and 800× magnification.

FIG. 5 shows image of hair swatches after exposure to the curl retention testing after application of leave-in hair serum containing serum base only, or serum base with either 1% silk polypeptide, 1% keratin ingredient, or 5% silicone elastomer ingredient.

FIG. 6 is a table of ingredients used in the wash-off shampoo formulation (not including the recombinant silk polypeptide and silicone elastomers) described in Example 6.

FIG. 7 is a table of ingredients used in the leave-on skin serum (not including the recombinant silk polypeptide and silicone elastomers) described in Example 7.

FIG. 8 is a table of ingredients used in the leave-on skin primer (not including the recombinant silk polypeptide and silicone elastomers) described in Example 8.

FIG. 9A is a graph of shear viscosity as a function of shear rate for the composition of FIG. 6 containing different amounts of recombinant silk polypeptide as compared to a placebo.

FIG. 9B is a chart showing the change in viscosity from placebo for the compositions tested in FIG. 9A.

FIGS. 10A and 10B are charts showing the change in rheology (G′ and G″) for the compositions tested in FIG. 9A.

FIG. 11 is a chart showing change in viscosity from placebo for compositions of FIG. 7 having different amounts of recombinant silk polypeptide.

FIG. 12A is a graph of shear viscosity as a function of shear rate for the composition of FIG. 8 containing different amounts of recombinant silk polypeptide as compared to a placebo.

FIG. 12B is a chart showing the change in viscosity from placebo for the compositions tested in FIG. 12A.

### DETAILED DESCRIPTION

The details of various embodiments of the invention are set forth in the description below. Other features, objects, and advantages of the invention will be apparent from the description and the drawings, and from the aspects.

This invention relates to the use of a recombinant silk polypeptide as a silicone replacement within a personal care and/or cosmetic composition for providing benefits to cosmetics, skin and hair that would normally be associated with linear silicones and silicone elastomers. The skin, hair or cosmetic compositions of the disclosure can have any standard ingredients typically included in such compositions, including active ingredients, pigments, and additives. The silicone replacement comprising a recombinant silk polypeptide can be used in combination with any standard hair care, skin care, or cosmetic ingredients to form compositions of the disclosure in which a silicone (fluid or elastomer) typically included in such compositions is replaced. Any skin, hair or cosmetic composition type is contemplated herein for use with the silicon elastomer replacement component, such as, but not limited to, SPF formulation, a color cosmetic, wash-off hair shampoo, skin serum, skin primer, or a hair serum. The recombinant silk polypeptide can deliver these benefits at equal or decreased loading levels compared to the silicone component. Advantageously, it has been observed that recombinant silk polypeptides as silicone replacements can maintain the G′ and G″ rheology curve shapes as inclusion of a silicone. Further, in some compositions, the recombinant silk polypeptide was found to significantly increase the viscosity of the formulation as compared to an equivalent amount of silicone, thereby allowing for significantly reduced loading levels of the recombinant silk polypeptide as compared to a silicone. Moreover the recombinant silk polypeptide is biodegradable and will degrade in the environment (for example, once washed down the drain), while the silicone component is not biodegradable.

Cosmetic, hair, or skin care compositions in accordance with the disclosure include a silicone replacement component comprising a recombinant silk polypeptide and one or more active ingredients for cosmetic, skin, or hair care. The compositions of the disclosure can be substantially free of silicone. Reference to “silicone” herein should be understood unless otherwise state to include both silicone fluid and silicone elastomer. For example, the compositions of the disclosure can have less than 0.1% silicone. For example, the composition of the disclosure can have less than 0.1% silicone elastomer.

Recombinant silk polypeptide is a high molecular weight polypeptide that has entropically self-assembled into a cross-linked and semicrystalline state. The recombinant polypeptide can be included in the composition as a powder. For example, hollow particles of recombinant silk polypeptide can be milled and included in the composition as a milled powder. The silicone replacement component can include the recombinant silk polypeptide present suspended in a solvent. The silicone replacement component can include the recombinant silk polypeptide as a randomly structure gel. At the macro-level this could range from a low viscosity weak gel suspended within an aqueous solvent (sometimes referred to as “slurry”), to a dry hollow powder particle (<15% moisture content).

In some embodiments, provided herein is a composition comprising recombinant silk polypeptide and use thereof as silicone replacements in beauty and personal care formulations.

In some embodiments, the recombinant silk polypeptide is a high molecular weight polypeptide greater than >100 amino acids in length and less than 90 kDa amino acids in length.

In some embodiments, the recombinant silk polypeptide is self-assembled into a semicrystalline state in which the crystalline portion is characterized by beta-sheet crosslinks that are resistant to solubility in water at pH from 3-8, other polar and non polar solvents (hexanol, hexane, benzene), oils, waxes, surfactants (anionic, non-ionic, cationic, amphoteric) but easily dispersed in these materials to form a heterogeneous dispersion.

In some embodiments, the recombinant silk polypeptide exists as a randomly structured gel. This gel can also include the presence of preservative and chelating agents. In various aspects, the recombinant silk polypeptide exists as a hollow powder. The hollow powder can be milled such that the powder incorporated into the composition as a milled powder. The powder can also include or be mixed with preservative and chelating agents.

In some embodiments, the recombinant silk polypeptide exists as a hollow powder suspended in an aqueous, polar, non-polar, oil, wax, or surfactant diluent. This mixture can also include the presence of preservative and chelating agents.

The recombinant silk polypeptide can be included in the composition in an amount of about 0.01 wt % to about 30 wt % based on the total weight of the composition. For example, the recombinant silk polypeptide can be included in the composition, based on the total weight of the composition, in an amount of about 0.05 wt % to about 5 wt %, about 0.5 wt % to about 5 wt %, about 0.01 wt % to about 0.5 wt %, about 0.1 wt % to about 0.5 wt %, about 0.05 wt % to about 5 wt %, about 0.5 wt % to about 5 wt %, about 5 wt % to about 20 wt %, about 5 wt % to about 30 wt %, about 25 wt % to about 30 wt %, about 10 wt % to about 25 wt %, or about 1 wt % to about 5 wt %.

In some embodiments, the recombinant silk polypeptide forms a distinctive and detectable film on the skin and hair when applied within a leave-on formulation or wash-off formulation.

In some embodiments, the recombinant silk polypeptide helps to cleanse or exfoliate when applied within a wash-off formula.

In some embodiments, the recombinant silk polypeptide can be detected within a formulation (leave-on or wash-off) as evidenced by visual inspection with microscopy where a powder can be observed at 5-100× objective. Additionally the recombinant silk polypeptide can be detected by a high molecular weight peak between 50 kDa and 90 kDa using SEC-HPLC.

In some embodiments, the recombinant silk polypeptide matches silicone elastomer performance in skincare, haircare, cosmetic, personal care, antiperspirant/deodorant formulations at the same concentration or less.

In some embodiments, the recombinant silk polypeptide outperforms silicone elastomer performance in skincare, haircare, cosmetic, personal care, antiperspirant/deodorant formulations at the same concentration or less.

In some embodiments, the recombinant silk polypeptide replaces the silicone elastomer in at least a 1:1 ratio up to a 1:60 ratio, meaning 1 parts silicone elastomer can be replaced with 1 parts recombinant silk polypeptide up to 60 parts silicone elastomer can be replaced with 1 parts recombinant silk polypeptide. For example, a shampoo was produced having a silk polypeptide as a silicone replacement, with the silk polypeptide being present in an amount of 0.05 wt % based on the total weight of the compositions, whereas the same composition required a silicon elastomer in an amount of 3 wt % (a 60× increase) to achieve the same performance.

In some embodiments, the performance characteristics of the composition include one or more of
a) Silky, smooth, and powdery feel
b) Decrease in glossiness on the skin
c) Enhanced shine on the hair
d) Vibrant and efficient pigment delivery
e) Easy spreadability
f) Quick absorption time
g) Mattification (i.e., less greasy after feel)
h) Wrinkle blurring effect
i) Style retention and heat resistance for hair
j) UV and pollution defense
k) Increased formulation viscosity

In some embodiments, the recombinant silk polypeptide is compatible (meaning its structure and performance is maintained) with a wide variety of common cosmetic components such as polar and non-polar solvents, oils, waxes, fatty acids, humectants, and (sunscreen) actives.

Due to the difference in recombinant silk polypeptide swelling in different solvents, these differences can be used as formulation processing aids. For example, recombinant silk polypeptide can be added to a formulation in the unswelled state within an oil, wax, or non-polar solvent, and then upon coming in contact with water will swell. The recombinant silk polypeptide is fully or partially removed from skin and hair by water and fully removed by surfactants.

In some embodiments, the recombinant silk polypeptide outperforms silicone elastomers for biodegradation in both anaerobic and aerobic digestion conditions. Within an Organisation for Economic Co-operation and Development (OECD) 301 ready test the recombinant silk polypeptide will experience a rapid biodegradation during the first 3-5 days of at least 5-15%. During the subsequent 5-90 days of incubation, the recombinant silk polypeptide will experience constant increasing biodegradation with an absence of a plateau greater than 20 days. OECD (1992), Test No. 301: Ready Biodegradability, OECD Guidelines for the Testing of Chemicals, Section 3, OECD Publishing, Paris, doi.org/10.1787/9789264070349-en, incorporated herein by reference in its entirety.

### Definitions

The following terms, unless otherwise indicated, shall be understood to have the following meanings:

The term “stability”, as used herein with respect to silk proteins, refers to the ability of the product not to form a gelation, discoloration or turbidity that is due to the self-aggregation of silk proteins. For example, U.S. Patent Publication No. 2015/0079012 (Wray et al.) is directed to the use of humectant, including glycerol to increase the shelf-stability of skincare products comprising full-length silk fibroin. U.S. Pat. No. 9,187,538 is directed to a skincare formulation comprising full-length silk fibroin that is shelf stable for up to 10 days. Both of these publications are incorporated herein by reference in their entirety.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO: 1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally occurring and non-naturally occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and aspects, the word “comprise” or variations such as “comprises” or “comprising,” will be understood to imply the inclusion of a stated integer or group of integers but not the exclusion of any other integer or group of integers.

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “powder” as used herein refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. Dry powder may be produced by spray-drying, lyophilization, and/or according to methods known in the art.

The term “carrier” refers to a recombinant protein used for surface hydration, surface cleansing, surface defense, surface detoxification, surface exfoliation, surface improvement, coloring, and/or delivery of various additives or solvents, including, but not limited to, water, glycerin, alcohols, siloxane, oils, humectants, emollients, occlusive agents, active agents, and/or cosmetic adjuvants to a surface like skin, hair, or nails. The carrier as used herein comprises an outer shell and hollow core, e.g., 18B protein.

The term “cosmetics” as used herein includes make-up, foundation, skin care, hair care, and nail care products.

The term “make-up” as used herein refers to products that leave color on the face, including foundation, blacks and browns, i.e., mascara, concealers, eye liners, brow colors, eye shadows, blushers, lip colors, powders, solid emulsion compact, and so forth.

The term “foundation” as used herein refers to liquid, cream, mousse, pancake, compact, concealer or like product created or reintroduced by cosmetic companies to even out the overall coloring of the skin.

The term “skin care products” as used herein refer to those used to treat or care for, or somehow moisturize, improve, or clean the skin. Products contemplated by the phrase “skin care products” include, but are not limited to, creams, mists, serums, cleansing gels, ampules, adhesives, patches, bandages, toothpaste, anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams, and the like.

The term “sagging” as used herein means the laxity, slackness, or the like condition of skin that occurs as a result of loss of, damage to, alterations to, and/or abnormalities in dermal elastin, muscle and/or subcutaneous fat.

The terms “treating” or “treatment” as used herein refer to the treatment (e.g., alleviation or elimination of symptoms and/or cure) and/or prevention or inhibition of the condition (e.g., a skin condition) or relief of symptoms.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1A lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 2871) is, for the purposes of the present invention, the same as GSGAG (SEQ ID NO: 2872) and the same as GGSGA (SEQ ID NO: 2873); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1A
Samples of Block Sequences

| Species | Silk Type | Representative Block Amino Acid Sequence |
|---|---|---|
| Aliatypus gulosus | Fibroin 1 | GAASSSSTIITTKSASASAAADASAAATASAASRSSANAAASAFAQS |
| | | FSSILLESGYFCSIFGSSISSSYAAAIASAASRAAAESNGYTTHAYA |
| | | CAKAVASAVERVTSGADAYAYAQAISDALSHALLYTGRLNTANANSL |
| | | ASAFAYAFANAAAQASASSASAGAASASGAASASGAGSAS(SEQ |
| | | ID NO: 2844) |
| Plectreurys tristis | Fibroin 1 | GAGAGAGAGAGAGAGAGSGASTSVSTSSSSGSGAGAGAGSGAGSGAG |
| | | AGSGAGAGAGAGGAGAGFGSGLGLGYGVGLSSAQAQAQAQAAAQAQA |
| | | QAQAQAYAAAQAQAQAQAQAQAAAAAAAAAAA (SEQ ID NO: |
| | | 2845) |
| Plectreurys tristis | Fibroin 4 | GAAQKQPSGESSVATASAAATSVTSGGAPVGKPGVPAPIFYPQGPLQ |
| | | QGPAPGPSNVQPGTSQQGPIGGVGGSNAFSSSFASALSLNRGFTEVI |
| | | SSASATAVASAFQKGLAPYGTAFALSAASAAADAYNSIGSGANAFAY |
| | | AQAFARVLYPLVQQYGLSSSAKASAFASAIASSESSGTSGQGPSIGQ |
| | | QQPPVTISAASASAGASAAAVGGGQVGQGPYGGQQQSTAASASAAAA |
| | | TATS(SEQ ID NO: 2846) |
| Araneus gemmoides | TuSp | GNVGYQLGLKVANSLGLGNAQALASSLSQAVSAVGVGASSNAYANAV |
| | | SNAVGQVLAGQGILNAANAGSLASSFASALSSSAASVASQSASQSQA |
| | | ASQSQAAASAFRQAASQSASQSDSRAGSQSSTKTTSTSTSGSQADSR |
| | | SASSSASQASASAFAQQSSASLSSSSSFSSAFSSATSISAV (SEQ |
| | | ID NO: 2847) |
| Argiope aurantia | TuSp | GSLASSFASALSASAASVASSAAAQAASQSQAAASAFSRAASQSASQ |
| | | SAARSGAQSISTTTTTSTAGSQAASQSASSAASQASASSFARASSAS |
| | | LAASSSESSAFSSANSLSALGNVGYQLGENVANNLGIGNAAGLGNAL |
| | | SQAVSSVGVGASSSTYANAVSNAVGQFLAGQGILNAANA (SEQ ID |
| | | NO: 2848) |
| Deinopis spinosa | TuSp | GASASAYASAISNAVGPYLYGLGLENQANAASFASSFASAVSSAVAS |
| | | ASASAASSAYAQSAAAQAQAASSAFSQAAAQSAAAASAGASAGAGAS |
| | | AGAGAVAGAGAVAGAGAVAGASAAAASQAAASSSASAVASAFAQSAS |
| | | YALASSSAFANAFASATSAGYLGSLAYQLGLTTAYNLGLSNAQAFAS |
| | | TLSQAVTGVGL (SEQ ID NO: 2849) |
| Nephila clavipes | TuSp | GATAASYGNALSTAAAQFFATAGLLNAGNASALASSFARAFSASAES |
| | | QSFAQSQAFQQASAFQQAASRSASQSAAEAGSTSSSTTTTTSAARSQ |
| | | AASQSASSSYSSAFAQAASSSLATSSALSRAFSSVSSASAASSLAYS |
| | | IGLSAARSLGIADAAGLAGVLARAAGALGQ (SEQ ID NO: |
| | | 2850) |
| Argiope trifasciata | Flag | GGAPGGGPGGAGPGGAGFGPGGGAGFGPGGGAGFGPGGAAGGPGGPG |
| | | GPGGPGGAGGYGPGGAGGYGPGGVGPGGAGGYGPGGAGGYGPGGSGP |
| | | GGAGPGGAGGEGPVTVDVDVTVGPEGVGGGPGGAGPGGAGFGPGGGA |
| | | GFGPGGAPGAPGGPGGPGGPGGPGGPGGVGPGGAGGYGPGGAGGVGP |
| | | AGTGGFGPGGAGGFGPGGAGGFGPGGAGGFGPAGAGGYGPGGVGPGG |
| | | AGGFGPGGVGPGGSGPGGAGGEGPVTVDVDVSV (SEQ ID NO: |
| | | 2851) |
| Nephila clavipes | Flag | GVSYGPGGAGGPYGPGGPYGPGGEGPGGAGGPYGPGGVGPGGSGPGG |
| | | YGPGGAGPGGYGPGGSGPGGYGPGGSGPGGYGPGGSGPGGYGPGGSG |
| | | PGGYGPGGYGPGGSGPGGSGPGGSGPGGYGPGGTGPGGSGPGGYGPG |
| | | GSGPGGSGPGGYGPGGSGPGGFGPGGSGPGGYGPGGSGPGGAGPGGV |
| | | GPGGFGPGGAGPGGAAPGGAGPGGAGPGGAGPGGAGPGGAGPGGAGP |
| | | GGAGGAGGAGGSGGAGGSGGTTIIEDLDITIDGADGPITISEELPIS |
| | | GAGGSGPGGAGPGGVGPGGSGPGGVGPGGSGPGGVGPGGSGPGGVGP |
| | | GGAGGPYGPGGSGPGGAGGAGGPGGAYGPGGSYGPGGSGGPGGAGGP |
| | | YGPGGEGPGGAGGPYGPGGAGGPYGPGGAGGPYGPGGEGGPYGP |
| | | (SEQ ID NO: 2852) |
| Latrodectus | AcSp | GINVDSDIGSVTSLILSGSTLQMTIPAGGDDLSGGYPGGFPAGAQPS |
| hesperus | | GGAPVDFGGPSAGGDVAAKLARSLASTLASSGVFRAAFNSRVSTPVA |
| | | VQLTDALVQKIASNLGLDYATASKLRKASQAVSKVRMGSDTNAYALA |
| | | ISSALAEVLSSSGKVADANINQIAPQLASGIVLGVSTTAPQFGVDLS |
| | | SINVNLDISNVARNMQASIQGGPAPITAEGPDFGAGYPGGAPTDLSG |
| | | LDMGAPSDGSRGGDATAKLLQALVPALLKSDVFRAIYKRGTRKQVVQ |
| | | YVTNSALQQAASSLGLDASTISQLQTKATQALSSVSADSDSTAYAKA |
| | | FGLAIAQVLGTSGQVNDANVNQIGAKLATGILRGSSAVAPRLGIDLS |
| | | (SEQ ID NO: 2853) |
| Argiope trifasciata | AcSp | GAGYTGPSGPSTGPSGYPGPLGGGAPFGQSGFGGSAGPQGGFGATGG |
| | | ASAGLISRVANALANTSTLRTVLRTGVSQQIASSVVQRAAQSLASTL |
| | | GVDGNNLARFAVQAVSRLPAGSDTSAYAQAFSSALFNAGVLNASNID |
| | | TLGSRVLSALLNGVSSAAQGLGINVDSGSVQSDISSSSSFLSTSSSS |
| | | ASYSQASASSTS(SEQ ID NO: 2854) |
| Uloborus diversus | AcSp | GASAADIATAIAASVATSLQSNGVLTASNVSQLSNQLASYVSSGLSS |
| | | TASSLGIQLGASLGAGFGASAGLSASTDISSSVEATSASTLSSSASS |
| | | TSVVSSINAQLVPALAQTAVLNAAFSNINTQNAIRIAELLTQQVGRQ |
| | | YGLSGSDVATASSQIRSALYSVQQGSASSAYVSAIVGPLITALSSRG |
| | | VVNASNSSQIASSLATAILQFTANVAPQFGISIPTSAVQSDLSTISQ |
| | | SLTAISSQTSSSVDSSTSAFGGISGPSGPSPYGPQPSGPTFGPGPSL |
| | | SGLTGFTATFASSFKSTLASSTQFQLIAQSNLDVQTRSSLISKVLIN |
| | | ALSSLGISASVASSIAASSSQSLLSVSA (SEQ ID NO: 2855) |
| Euprosthenops | MaSp1 | GGQGGQGQGRYGQGAGSSAAAAAAAAAAAAAA (SEQ ID NO: |
| australis | | 2856) |
| Tetragnatha | MaSp1 | GGLGGGQGAGQGGQQGAGQGGYGSGLGGAGQGASAAAAAAAA (SEQ |
| kauaiensis | | ID NO: 2857) |
| Argiope aurantia | MaSp2 | GGYGPGAGQQGPGSQGPGSGGQQGPGGLGPYGPSAAAAAAAA (SEQ |
| | | ID NO: 2858) |
| Deinopis spinosa | MaSp2 | GPGGYGGPGQQGPGQGQYGPGTGQQGQGPSGQQGPAGAAAAAAAAA |
| | | (SEQ ID NO: 2859) |
| Nephila clavata | MaSp2 | GPGGYGLGQQGPGQQGPGQQGPAGYGPSGLSGPGGAAAAAAA (SEQ |
| | | ID NO: 2860) |
| Deinopis Spinosa | MiSp | GAGYGAGAGAGGGAGAGTGYGGGAGYGTGSGAGYGAGVGYGAGAGA |
| | | GGGAGAGAGGGTGAGAGGGAGAGYGAGTGYGAGAGAGGGAGAGAGA |
| | | GAGAGAGAGSGAGAGYGAGAGYGAGAGAGGVAGAGAAGGAGAAGGA |
| | | GAAGGAGAAGGAGAGAGAGSGAGAGAGGGARAGAGG (SEQ ID |
| | | NO: 2861) |
| Latrodectus | MiSp | GGGYGRGQGAGAGVGAGAGAAAGAAAIARAGGYGQGAGGYGQGQGA |
| hesperus | | GAAAGAAAGAGAGGYGQGAGGYGRGQGAGAGAGAGAGARGYGQGAG |
| | | AGAAAGAAASAGAGGYGQGAGGYGQGQGAGAAAGAAASAGAGGYGQ |
| | | GAGGYGQGQGA (SEQ ID NO: 2862) |
| Nephila clavipes | MiSp | GAGAGGAGYGRGAGAGAGAAAGAGAGAAAGAGAGAGGYGGQGGYGA |
| | | GAGAGAAAAAGAGAGGAAGYSRGGRAGAAGAGAGAAAGAGAGAGGY |
| | | GGQGGYGAGAGAGAAAAAGAGSGGAGGYGRGAGAGAAAGAGAAAGA |
| | | GAGAGGYGGQGGYGAGAGAAAAA (SEQ ID NO: 2863) |
| Nephilengys | MiSp | GAGAGVGGAGGYGSGAGAGAGAGAGAASGAAAGAAAGAGAGGAGGY |
| cruentata | | GTGQGYGAGAGAGAGAGAGGAGGYGRGAGAGAGAGAGGAGGYGAGQ |
| | | GYGAGAGAGAAAAAGDGAGAGGAGGYGRGAGAGAGAGAAAGAGAGG |
| | | AGGYGAGQGYGAGAGAGAAAGAGAGGAGGYGAGQGYGAGAGAGAAA |
| | | AA (SEQ ID NO: 2864) |
| Uloborus diversus | MiSp | GSGAGAGSGYGAGAGAGAGSGYGAGSSASAGSAINTQTVTSSTTTS |
| | | SQSSAAATGAGYGTGAGTGASAGAAASGAGAGYGGQAGYGQGAGAS |
| | | ARAAGSGYGAGAGAAAAAGSGYGAGAGAGAGSGYGAGAAA (SEQ |
| | | ID NO: 2865) |
| Uloborus diversus | MiSp | GAGAGYRGQAGYIQGAGASAGAAAAGAGVGYGGQAGYGQGAGASAG |
| | | AAAAAGAGAGRQAGYGQGAGASAGAAAAGAGAGRQAGYGQGAGASA |
| | | GAAAAGADAGYGGQAGYGQGAGASAGAAASGAGAGYGGQAGYGQGA |
| | | GASAGAAAAGAGAGYLGQAGYGQGAGASAGAAAGAGAGYGGQAGYG |
| | | QGTGAAASAAASSA (SEQ ID NO: 2866) |
| Araneus | MaSp1 | GGQGGQGGYGGLGSQGAGQGGYGAGQGAAAAAAAAGGAGGAGRGGL |
| ventricosus | | GAGGAGQGYGAGLGGQGGAGQAAAAAAAGGAGGARQGGLGAGGAGQ |
| | | GYGAGLGGQGGAGQGGAAAAAAAAGGQGGQGGYGGLGSQGAGQGGY |
| | | GAGQGGAAAAAAAAGGQGGQGGYGGLGSQGAGQGGYGGRQGGAGAA |
| | | AAAAAA (SEQ ID NO: 2867) |
| Dolomedes | MaSp1 | GGAGAGQGSYGGQGGYGQGGAGAATATAAAAGGAGSGQGGYGGQGG |
| tenebrosus | | LGGYGQGAGAGAAAAAAAAAGGAGAGQGGYGGQGGQGGYGQGAGAG |
| | | AAAAAAGGAGAGQGGYGGQGGYGQGGGAGAAAAAAAASGGSGSGQG |
| | | GYGGQGGLGGYGQGAGAGAGAAASAAAA (SEQ ID NO: 2868) |
| Nephilengys | MaSp | GGAGQGGYGGLGGQGAGAAAAAAGGAGQGGYGGQGAGQGAAAAAAS |
| cruentata | | GAGQGGYEGPGAGQGAGAAAAAAGGAGQGGYGGLGGQGAGQGAGAA |
| | | AAAAGGAGQGGYGGLGGQGAGQGAGAAAAAAGGAGQGGYGGQGAGQ |
| | | GAAAAAAGGAGQGGYGGLGSGQGGYGRQGAGAAAAAAAA (SEQ |
| | | ID NO: 2869) |
| Nephilengys | MaSp | GGAGQGGYGGLGGQGAGAAAAAAGGAGQGGYGGQGAGQGAAAAAAS |
| cruentata | | GAGQGGYGGPGAGQGAGAAAAAAGGAGQGGYGGLGGQGAGQGAGAA |
| | | AAAAGGAGQGGYGGQGAGQGAAAAAAGGAGQGGYGGLGSGQGGYGG |
| | | QGAGAAAAAGGAGQGGYGGLGGQGAGQGAGAAAAAA (SEQ ID |
| | | NO: 2870) |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3×FLAG nucleotide coding sequence. In some embodiments, the silk polypeptide nucleotide coding sequence is operatively linked to other affinity tags such as 6-8 His residues.

In some embodiments, the recombinant silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant silk protein comprises repeat units wherein each repeat unit has at least 95% sequence identity to a sequence that comprises from 2 to 20 quasi-repeat units; each quasi-repeat unit comprises {GGY-[GPG-X₁]ₙ₁-GPS-(A)ₙ₂}, wherein for each quasi-repeat unit; X₁ is independently selected from the group consisting of SGGQQ (SEQ ID NO: 2874), GAGQQ (SEQ ID NO: 2875), GQGOPY (SEQ ID NO: 2876), AGQQ (SEQ ID NO: 2877), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant silk polypeptide comprises the polypeptide sequence of SEQ ID NO: 2878 (i.e., 18B). In some embodiments, the repeat unit is a polypeptide comprising SEQ ID NO: 2879. These sequences are provided in Table 1B:

TABLE 1B
Exemplary polypeptides sequences of recombinant protein and repeat unit

| SEQ ID | Polypeptide Sequence |
|---|---|
| SEQ ID NO: 2878 | GGYGPGAGQQGPGSGGQQGPGGQGPYGSGQQGPGGAGQQGPGGQGPYGPGAAAAAAAAAG |
| | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ ID NO: 2879 | GGYGPGAGQQGPGSGGQQGPGGQGPYGSGQQGPGGAGQQGPGGQGPYGPGAAAAAAAAAG |
| | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAA |

In some embodiments, the structure of fibers formed from the described recombinant silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. Without intending to be limited by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of recombinant protein fibers (RPFs). For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; adiptic acid; amide of dimethylaminopropyl amine and caprylic/capric acid; acetamide; and any combination thereof.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant silk polypeptides are produced by fermentation and recovered as recombinant silk polypeptide powder from the same, there may be impurities present in the recombinant silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant silk polypeptide powder or composition. Size Exclusion Chromatography separates molecules based on their relative size and can be used to analyze the relative amounts of recombinant silk polypeptide in its full-length polymeric and monomeric forms as well as the amount of high, low and intermediate molecular weight impurities in the recombinant silk polypeptide powder. Similarly, Rapid High Performance Liquid Chromatography may be used to measure various compounds present in a solution such as monomeric forms of the recombinant silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant silk polypeptide may have a purity calculated based on the amount of the recombinant silk polypeptide in its monomeric form by weight relative to the other components of the recombinant silk polypeptide powder. In various instances, the purity can range from 50% by weight to 90% by weight, depending on the type of recombinant silk polypeptide and the techniques used to recover, separate and post-process the recombinant silk polypeptide powder.

Both Size Exclusion Chromatography and Reverse Phase High Performance Liquid Chromatography are useful in measuring full-length recombinant silk polypeptide, which makes them useful techniques for determining whether processing steps have degraded the recombinant silk polypeptide by comparing the amount of full-length silk polypeptide in a composition before and after processing. In various embodiments of the present invention, the amount of full-length recombinant silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Silicone Replacement Component

The silicone replacement component includes the recombinant silk polypeptide. The silicone replacement component can consists of the recombinant silk polypeptide. The silicone replacement component can include the recombinant silk polypeptide with a solvent and/or one or more additives, such as preservatives and chelating agents. The silicone replacement component can include the recombinant silk polypeptide in an amount, based on the total weight of the silicone replacement component, of about 1 wt % to about 40 wt % or in any other suitable amount needed to achieve a final desired loading the recombinant silk polypeptide in the cosmetic, skin or hair care composition.

Without intending to be limited by theory, in various embodiments of the present invention, inducing the silicone replacement Component may be used in applications where it is desirable to prevent the aggregation of the monomeric recombinant silk polypeptide into its crystalline polymeric form or to control the transition of the recombinant silk polypeptide into its crystalline polymeric form at a later stage in processing. In other embodiments, such inducing is not required.

In one specific embodiment, the silicon elastomer replacement component may be used to prevent aggregation of the recombinant silk polypeptide prior to blending the recombinant silk polypeptide with a second polymer. In another specific embodiment, the silicon elastomer replacement component may be used to create a base for a cosmetic or skincare product where the recombinant silk polypeptide is present in the base in its monomeric form. In this embodiment, having the recombinant silk polypeptide in its monomeric form in a base allows for the controlled aggregation of the monomer into its crystalline polymeric form upon contact with skin or through various other chemical reactions.

In various embodiments, the temperature to which the silicon elastomer replacement component having the recombinant silk polypeptide is heated will be minimized in order to minimize or entirely prevent degradation of the recombinant silk polypeptide. In specific embodiments, the recombinant silk melt will be heated to a temperature of less than 120° C., less than 100° C., less than 80° C., less than 60° C., less than 40° C., or less than 20° C. Often the melt will be at a temperature in the range 10° C. to 120° C., 10° C. to 100° C., 15° C. to 80° C., 15° C. to 60° C., 18° C. to 40° C. or 18° C. to 22° C. during processing. In other embodiments, the silicon elastomer replacement component is not heated. In such embodiments, the presence of heat is not required to form a silicon elastomer replacement component.

The amount of degradation of the recombinant silk polypeptide may be measured using various techniques. As discussed above, the amount of degradation of the recombinant silk polypeptide may be measured using Size Exclusion Chromatography to measure the amount of full-length recombinant silk polypeptide present. In various embodiments, the recombinant silk polypeptide is degraded in an amount of less than 6.0 weight % after it is formed into a molded body. In another embodiment, the recombinant silk polypeptide is degraded in an amount of less than 4.0 weight % after molding, less than 3.0 weight %, less than 2.0 weight %, or less than 1.0 weight %, such that the amount of degradation may be in the range 0.001% by weight to 10%, 8%, 6%, 4%, 3%, 2% or 1% by weight, or 0.01% by weight to 6%, 4%, 3%, 2% or 1% by weight. In another embodiment, the recombinant silk protein in the composition is substantially non-degraded. In a similar embodiment, the recombinant silk protein in the composition is substantially non-degraded over a period of time, at least 1 day, 1 month, 1 year, or 5 years.

In some embodiments, the silicone replacement component is physically stable. In various embodiments, the component remains in its material form, e.g., a powder, for a prolonged period of time, with a prolonged shelf life. On prolonged use, the silicone replacement component remains substantially stable. In some embodiments, the silicone replacement component has stability substantially the same as the stability of a silicone and/or silicone elastomer.

In some embodiments, the silicone replacement component has material properties substantially similar to the material properties of a silicone and/or silicone elastomer. In various embodiments, the silicone replacement component has substantially similar rheology as a silicone and/or silicone elastomer and/or imparts to a composition of the disclosure similar rheology as inclusion of a silicone and/or silicone elastomer.

In most embodiments of the present invention, the silicone replacement component is in a powder form. The silicone replacement component can include the recombinant silk polypeptide in a powder. In some embodiments, the silicone replacement component is spray-dried. In other embodiments, the silicone replacement component is freeze-dried or vacuum-dried. The terms “spray-drying” and “spray-dried” are used herein for simplicity but the skilled person will appreciate that freeze-drying or lyophilization and vacuum drying can be substituted for spray-drying as appropriate. These silicone replacement components may be stored dry.

The 18B protein is more stable in a dried form than in an aqueous slurry. In some embodiments, spray-dried recombinant silk is obtained as follows: a slurry composition comprising extracted recombinant silk is kept chilled during the drying step. It is pumped to a tall form spray dryer where the moisture content of the resulting powder is tightly controlled. As the protein powder is hydroscopic, the final powder collection and packout is performed to minimize reintroduction of moisture. The design of the packaging material should minimize moisture and light exposure.

In some embodiments, recovery and separation of the recombinant silk polypeptide from a cell culture is performed as follows: i) extraction and separation, ii) urea removal by ultrafiltration, iii) washing by precipitation, iv) salt removal and protein concentration, and v) spray drying.

In some embodiments, to freeze-dry a composition it is cooled until it solidifies and placed under reduced pressure to cause the most volatile ingredients in the composition to sublime. The solid residue may form a single mass which requires milling to form a fine powder. A typical freeze-dried powder comprises porous irregular shaped particles and readily hydrates. As freeze-drying does not require strong heat it is used to produce powders which comprise volatile ingredients. In some embodiments, the silicone replacement component is deep freeze-dried at a temperature below about −100° C.

After formation of the silicone replacement component, the crystallinity of the silicone replacement component can increase, thereby strengthening the composition. In some embodiments, the silicone replacement component stays the same or decreases. In some embodiments, the crystallinity index of the silicone replacement component as measured by X-ray crystallography is from 2% to 90%. In some other embodiments, the crystallinity index of the silicone replacement component as measured by X-ray crystallography is at least 3%, at least 4%, at least 5%, at least 6%, or at least 7%.

In some embodiments of the present invention, the silicone replacement component is a solid or film. In some embodiments, the silicone replacement component is a powder. In some embodiments, the solid or film will be substantially homogeneous meaning that the material, as inspected by light microscopy, has a low amount or does not have any inclusions or precipitates. In some embodiments, light microscopy may be used to measure birefringence which can be used as a proxy for alignment of the recombinant silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant silk solids and films will have minimal birefringence. In various embodiments, the solid is a bead. In some other embodiments, the solid functions as an exfoliant. The recombinant silk solid may be in the form of a gentle skin scrub for the skin. In some embodiments, the material form is a roll, pellet, sheet, or flake.

In some embodiments, the recombinant silk protein comprises a hollow core and/or a shell. In some embodiments, the recombinant silk protein ranges from about 1 μm to about 30 μm in diameter, about 5 μm to about 20 μm, or about 10 μm to about 50 μm in diameter, while recombinant silk protein in water ranges from about 20 to about 80 μm in diameter, about 30 μm to about 70 μm, or about 40 μm to about 100 μm in diameter. Prior to incorporate into the compositions of the disclosure, the recombinant silk protein hollow powder can be milled and incorporated as a milled powder.

### Solvents

In some embodiments, the silicone replacement component can include one or more solvents. For example, the recombinant silk polypeptide can be suspended in a solvent. The solvent can be an aqueous solvent, an alcohol, or an oil-based solvent. For example, the solvent can be one or more of water, glycerin, deionized water, olive oil, and pentylene glycol. For example, the recombinant silk polypeptide can be treated with a solvent such that the hollow core contains the solvent such as liquid water or glycerin, either in form of liquid water itself, or as a liquid aqueous solution, as an emulsion containing liquid water, or as an aqueous dispersion. In some embodiments, the silicone replacement component comprises about a 25 wt % solution in glycerin.

In some embodiments, the solvent is water. Without intending to be limited by theory, subjecting the recombinant silk polypeptide to a solvent such as water results in a recombinant silk polypeptide that has expanded or swelled, wherein the protein functions as a carrier containing the solvent (e.g., water). These compositions can be stored dry and partially rehydratable after immersion in water to directly form a liquid or semi-liquid aqueous suspension of expanded particles.

In some embodiments, the recombinant silk protein may expand a portion of the hollow core. In some other embodiments, the recombinant silk protein may expand a portion of the shell. In such embodiments where the solvent is water, the recombinant silk protein transforms into a hydrogel. In other embodiments where the solvent is water, the recombinant silk protein transforms into a paste. In various embodiments, heat and/or pressure may be added to further process the recombinant silk protein compositions.

In some embodiments, a solvent is generally present in a proportion ranging from 55 to 90% by weight relative to the total weight of the recombinant silk polypeptide. This range includes all specific values and subranges there between, including 60%, 65%, 70%, 75%, 80%, and 85% by weight. In some embodiments, the recombinant silk protein is insoluble in various solvents, including water at various different pH levels, glycerin, alcohols, siloxane, and oils.

In some embodiments, the solvent is an aqueous type. In such embodiments, the solvent is water. The solvent may have a pH ranging from 6 to 12. In some embodiments, the solvent has a pH of 6. In some other embodiments, the solvent has a pH ranging from 0 to 5, from 2 to 7, from 4 to 9, from 6 to 11, from 8 to 13, or from 10 to 14.

In other embodiments, the solvent includes a mixture of various volatile organic solvents, in order to obtain relatively short drying times. In some embodiments, the solvent is an alcohol.

Solvents may include water, ethyl alcohol, toluene, methylene chloride, isopropanol, n-butyl alcohol, castor oil, organopolysiloxane oils, ethylene glycol monoethyl ether, diethylene glycol monobutyl ether, diethylene glycol monoethyl ether, dimethyl sulphoxide, dimethyl formamide and tetrahydrofuran.

In some embodiments, the organopolysiloxane oil may be volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Suitable silicones are disclosed in U.S. Pat. No. 5,069,897, issued Dec. 3, 1991, which is incorporated by reference herein in its entirety. Examples of suitable organopolysiloxanes include, but are not limited to, polyalkylsiloxanes, alkyl substituted dimethicones, dimethiconols, polyalkylaryl siloxanes, and mixtures thereof. For instance, polyalkylsiloxanes, dimethicones and cyclomethicones may be used.

In some embodiments, the solvent is a vegetable oil and hydrogenated vegetable oil. In some embodiments, the solvent is a free fatty acid. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof. Animal fats and oils, e.g., cod liver oil, lanolin and derivatives thereof such as acetylated lanolin and isopropyl lanolate, may be used. Also useful are C₄-C₂₀ alkyl ethers of polypropylene glycols, C₁-C₂₀ carboxylic acid esters of polypropylene glycols, and di-C₈-C₃₀ alkyl ethers, examples of which include PPG-14 butyl ether, PPG-15 stearyl ether, dioctyl ether, dodecyl octyl ether, and mixtures thereof.

The compositions of the present invention may be substantially free of semi-solid hydrocarbons such as petrolatum, lanolin and lanolin derivatives, sterols (e.g., ethoxylated soya sterols), high molecular weight polybutenes and cocoa butter. By “substantially free,” as used herein, means that the concentration of the semi-solid hydrocarbons is less than 10%, or less than 5% or less than 2% or 0%.

### Recombinant Silk Proteins as a Cosmetics Formulation

In various embodiments, the recombinant silk protein is compounded into a silk cosmetic or skincare product (e.g., solutions applied to the skin or hair). Specifically, the recombinant silk protein can be incorporated into a silicone replacement component to be used as a base for a cosmetic or skincare product where the recombinant silk polypeptide is present in the base in its monomeric or less-crystalline form. In some embodiments, the silicone replacement component may be used as a base for a cosmetic or skincare product where the recombinant silk polypeptide is present in the base in a semi-crystalline form. In such embodiments, the recombinant silk polypeptide is not present in the base in its monomeric form.

In most embodiments, the cosmetic formulations are physically stable. In such embodiments, the recombinant silk protein and any other ingredients remain in its formulation for a prolonged period of time, with a prolonged shelf life. On prolonged use, the silicone replacement component remains substantially stable and the ingredients do not precipitate out of the formulation.

The composition of the invention may be used to apply the silk protein to the skin, nails, hair or mucous membranes, by contacting the composition with the skin, nails, hair or mucous membranes of a subject. Preferably, the inventive composition is used with human subjects.

In most embodiments, the cosmetic formulations are non-toxic or non-allergenic to subject hosts to which the cosmetic is applied. It is also desirable in the art to produce cosmetic compositions for hair and epidermal contact which will not permanently stain tissue and which can be removed by ordinary washing with aqueous detergents.

The solids, films, emulsions, hydrogels, and other material forms discussed in various embodiments may contain various humectants, emollients, occlusive agents, active agents, and cosmetic adjuvants, depending on the embodiment and the desired efficacy of the formulation. In some embodiments, the recombinant silk protein functions as a carrier. In some embodiments, the recombinant silk protein is a carrier, delivering one or more agents to a surface such as skin, hair, or nails.

In some embodiments, the cosmetic formulation comprises a plasticizer. Suitable concentrations of plasticizer by weight in the composition ranges from, e.g.: 1 to 60% by weight, 10 to 60% by weight, 10 to 50% by weight, 10 to 40% by weight, 15 to 40% by weight, 10 to 30% by weight, or 15 to 30% by weight. In some embodiments, the plasticizer is glycerol. In some embodiments, the plasticizer is triethanolamine, trimethylene glycol, polyethylene glycol, propylene glycol, sorbitol, sucrose, a saturated fatty acid, or an unsaturated fatty acid.

In the instance where water is used as a plasticizer, a suitable concentration of water by weight in the composition ranges from, e.g.: 5 to 80% by weight, 15 to 70% by weight, 20 to 60% by weight, 25 to 50% by weight, 19 to 43% by weight, or 19 to 27% by weight. Where water is used in combination with another plasticizer, it may be present in a range of, e.g., 5 to 50% by weight, 15 to 43% by weight or 19 to 27% by weight.

In some embodiments, suitable plasticizers may include polyols (e.g., glycerol), water, lactic acid, ascorbic acid, phosphoric acid, ethylene glycol, propylene glycol, triethanolamine, acid acetate, propane-1,3-diol or any combination thereof. In various embodiments, the amount of plasticizer can vary according to the purity and relative composition of the recombinant silk protein. For example, a higher purity powder may have less impurities such as a low molecular weight compound that may act as a plasticizer and therefore require the addition of a higher percentage by weight of plasticizer.

In some embodiments, the composition comprises a humectant or emollient. The term “humectant” as used herein refers to a hygroscopic substance that forms a bond with water molecules. Suitable humectants include, but are not limited to glycerol, propylene glycol, polyethylene glycol, pentalyene glycol, tremella extract, sorbitol, dicyanamide, sodium lactate, hyaluronic acid, aloe vera extract, alpha-hydroxy acid and pyrrolidonecarboxylate (NaPCA).

The term “emollient” as used herein refers to a compound that provide skin a soft or supple appearance by filling in cracks in the skin surface. Suitable emollients include, but are not limited to shea butter, cocao butter, squalene, squalane, octyl octanoate, sesame oil, grape seed oil, natural oils containing oleic acid (e.g., sweet almond oil, argan oil, olive oil, avocado oil), natural oils containing gamma linoleic acid (e.g., evening primrose oil, borage oil), natural oils containing linoleic acid (e.g., safflower oil, sunflower oil), or any combination thereof.

In some instances, an emollient or humectant may be an occlusive agent, and the disclosure contemplates inclusion of an occlusive agent into the composition in various embodiments. The term “occlusive agent” refers to a compound that forms a barrier on the skin surface to retain moisture. Other suitable occlusive agents may include, but are not limited to beeswax, canuba wax, ceramides, vegetable waxes, lecithin, allantoin. Without intending to be limited by theory, the film-forming capabilities of the silicone replacement component presented herein make an occlusive agent that forms a moisture retaining barrier because the recombinant silk polypeptides act attract water molecules and also act as humectants.

Optionally, the cosmetic formulation comprises an active agent. The term “active agent” refers to any compound that has a known beneficial effect in a hair care, skincare, or cosmetic formulation, including pigment in cosmetic formulations. Various active agents include, but are not limited to, acetic acid (i.e., vitamin C), alpha hydroxyl acids, beta hydroxyl acids, zinc oxide, titanium dioxide, retinol, niacinamide, other recombinant proteins (either as full length sequences or hydrolyzed into subsequences or “peptides”), copper peptides, curcuminoids, glycolic acid, hydroquinone, kojic acid, l-ascorbic acid, alpha lipoic acid, azelaic acid, lactic acid, ferulic acid, mandelic acid, dimethylaminoethanol (DMAE), resveratrol, natural extracts containing antioxidants (e.g. green tea extract, pine tree extract), caffeine, alpha arbutin, coenzyme Q-10, and salicylic acid.

The term “cosmetic adjuvant” refers to various other agents used to create a cosmetic product with commercially desirable properties, including, without limitation, surfactants, emulsifiers, preserving agents and thickeners.

As described herein, in various embodiments, the recombinant silk protein may form a semi-solid or gel-like structure that is dispersible. In various embodiments where the recombinant silk protein is compounded into a skin care formulation, the recombinant silk protein may form a non-reversible three-dimensional structure such as a gel or film that transforms into a dispersible liquid upon the surface of the skin.

In various embodiments, the recombinant silk protein may be suspended in water (“aqueous suspended protein”) to form a silicone replacement component in the form of a film, gel, or base that can be incorporated (i.e., compounded) in a cosmetic or skincare formulation. Depending on the embodiment, the amount of recombinant silk protein to water in the aqueous suspended protein can vary, as can the relative ratio of recombinant silk polypeptide powder to additive in the recombinant silk protein. In some embodiments, the silicone replacement component will comprise 10-33% recombinant silk polypeptide powder by weight. In some embodiments, a different solvent than water will be used. In some embodiments, the recombinant silk protein is suspended in water to create an aqueous suspended protein that is 1-40% recombinant silk protein and 60-99% water. In a specific embodiment, the silicone replacement component is suspended in water to create an aqueous suspended protein that is 10% recombinant silk polypeptide powder by weight, 30% additive by weight and 60% water by weight based on the total weight of the silicone replacement component. In a specific embodiment, the protein is suspended in water to create an aqueous suspended protein that is 6% recombinant silk polypeptide powder by weight, 18% additive by weight and 76% water by weight based on the total weight of the silicone replacement component. In a specific embodiment, the protein is suspended in water to create an aqueous suspended protein that is 10% recombinant silk polypeptide powder by weight and 90% water by weight based on the total weight of the silicone replacement component.

Depending on the embodiment, the aqueous suspended protein may be optionally heated and agitated when it is re-suspended in water. In some embodiments, heating and agitating the aqueous suspended protein may result in a phase transformation of the recombinant silk polypeptides in the aqueous suspended protein. Specifically, heating and agitating the aqueous suspended protein results in three distinct phases that are assessed by centrifugation: 1) a gel phase that is distinct from the supernatant after centrifugation; 2) a colloidal phase that can be filtered from the supernatant after centrifugation; and 3) a solution phase that remains after filtering the colloidal phase from the supernatant. Various combinations of heat, agitation and centrifugation may be used, provided that the aqueous suspended protein must not be subject to prolonged heat in order to prevent degradation of the recombinant silk polypeptides. In a specific embodiment, the protein is subjected to gentle agitation at 90° C. for 5 minutes and centrifuged at 16,000 RCF for 30 minutes.

In various embodiments, either the various phases of the aqueous suspended protein (i.e., colloidal phase, gel phase and solution) or the aqueous suspended protein may be incorporated in a cosmetic or skincare formulation to provide a source of recombinant silk protein. Depending on the embodiment, the aqueous suspended protein may be subject to agitation with or without heat before incorporating into a skincare formulation. Optionally, the aqueous suspended protein may be separated in the above-discussed phases by centrifugation and/or filtering. Depending on the embodiment, the skincare formulation may be an emulsion (e.g., a cream or serum) or a primarily aqueous solution (e.g., a gel). In certain embodiments, the recombinant silk protein may be incorporated into any of the cosmetic, skin care, or hair care formulations described herein without aqueous resuspension. In these compositions, a homogenizer or similar equipment may be used to ensure that the recombinant silk protein is uniformly distributed in the composition.

In some embodiments, the aqueous suspended protein may be subject to heat and agitation, then cast onto a flat surface and dried into a film. In some embodiments, the aqueous suspended protein may be cast onto a flat surface and dried into a film without being subjected to heat and/or agitation. In such embodiments, the aqueous suspended protein may be cast onto a flat surface and dried into a film without being subjected to additional processing. In some embodiments, the aqueous suspended protein may be incorporated into an emulsion, then cast onto a flat surface and dried into a film. Depending on the embodiment, various different drying conditions may be used. Suitable drying conditions include drying at 60° C. or at 80° C. with and without a vacuum. In embodiments that use a vacuum, 15 Hg is a suitable amount of vacuum. Other methods of drying are well established in the art.

In various embodiments, the films comprising the aqueous suspended protein alone have a low melting temperature. In various embodiments, the films comprising the aqueous suspended protein alone have melting temperature that is less than body temperature (around 34-36° C.) and melts upon contact with skin. Without intending to be limited by theory, the recombinant silk polypeptide forms enough intermolecular interactions to make a semi-solid structure (i.e., film); however this structure is reversible upon skin contact and can be re-formed after dispersion on the skin surface. In various embodiments, the film will have reduced crystallinity compared to the recombinant silk protein or the recombinant silk powder, as measured by Fourier-transform infrared spectroscopy (FTIR). In various embodiments, the films comprising the aqueous suspended protein do not melt upon contact with skin. In such embodiments, the film functions as a barrier. In various embodiments, the film is a hydrophobic film of low density. The film or barrier may range from about 1 μm to about 50 μm in thickness, from about 10 μm to about 30 μm, or from about 20 μm to about 40 μm in thickness. Upon contact with skin, the barrier may be formed on the surface of the epidermal layer, materializing a robust, non-specific adherence is made to the skin surface. In some embodiments, the thickness of the film changes depending on the concentration of recombinant silk protein and surface area of application.

In some embodiments, the barrier is long-lasting and prevents against one or more environmental stressors, including wind, humidity, harsh additives, pollution, abrasion, dirt, and grease. The barrier may withstand abrasion equivalent to at least 100 rubs by hand, at least 200 rubs, at least 400 rubs, at least 600 rubs, or at least 800 rubs.

In one specific embodiment, the aqueous suspended protein or the protein may be incorporated (e.g., homogenized) into an emulsion, then cast on a flat surface and lyophilized to create a porous film. Depending on the embodiment, various techniques may be used for lyophilization, including freezing the film at −80° C. for 30 minutes. Other lyophilization techniques will be well known to those skilled in the art.

In various embodiments, the above-described films can be used as a topical skincare agent. This film may be applied directly to the skin and can be re-hydrated to form a dispersible viscous substance that is incorporated into the skin. As discussed herein, various emollients, humectants, active agents, and other cosmetic adjuvants may be incorporated into the film. This film may be applied directly to the skin and adsorb to the skin due to contact with the skin, or after gently rubbing the film into the skin. In some embodiments, the film may be applied directly the skin and adsorb to the skin without additional rubbing or contact. In some embodiments, the protein resuspended in an aqueous solution may be applied to the face and then exposed to a coagulant such as propylene glycol via mist to form a gellable mask.

Depending on the embodiment, the film that is cast may be a flat film (i.e., with no surface variability) or may be cast on a mold that incorporates microstructures. In a specific embodiment, the film that is cast on a mold that incorporates microneedle structures to prick the surface of the skin and assist in delivery of active agents.

In an alternate embodiment, the aqueous suspended protein may be added to an emulsion that is used as a cosmetic product. The emulsion may be applied to skin or hair and then allowed to form a film on the surface of the skin upon drying. As discussed herein, various emollients, humectants, active agents, and other cosmetic adjuvants may be incorporated into the emulsion.

In some embodiments, the compositions of the disclosure may be liquid or semi-solid, such as creams, lotions, and gels. The compositions useful in the subject invention may be made into a wide variety of product forms that are known in the art. These include, but are not limited to, powders, lotions, creams, gels, patches, serums, ampules, powders, sticks, sprays, ointments, pastes, mousses, ointments, liquids, emulsions, foams, or aerosols. These product forms may comprise several types of additives, as further discussed herein, including, but not limited to, solutions, aerosols, emulsions, gels, solids, and liposomes. The compounds which are active in the compositions and methods of this invention may be delivered topically by any means known to those of skill in the art.

In some other embodiments, the compositions may be basic cosmetic compositions such as facial cleansers, such as toilet water, cream, essence, cleansing foam and cleansing water; pack and body oil; color cosmetic compositions such as foundation, lipstick, mascara, and make-up base; hair product compositions such as shampoo, rinse, hair conditioner and hair gel; soap; and the like. The cosmetic formulation can be prepared in any method known in the art, using the silicone replacement component described herein, optionally together with at least one carrier and/or additive, which are commonly used in the field of preparing cosmetic compositions.

In some embodiments, the compositions comprise at least one cosmetic agent. Examples of cosmetic agents include emollients, humectants, colorants, pigments, fragrances, moisturizers, viscosity modifiers and any other cosmetic forming agent. One or more cosmetic agents can be included in the cosmetic composition. In another embodiment, additional active ingredients as known in the art and described herein may also be used, including, but not limited to, a skin softener, a skin permeation enhancer, a colorant, an aromatic, an emulsifier, and a thickener. Also, the cosmetic composition may further comprise a perfumery, a pigment, a bactericidal agent, an antioxidant, a preservative, and/or a moisturizer, as well as inorganic salts and synthetic polymer substances, for, e.g., the purpose of improving physical properties.

The composition may also be delivered topically via a lotion. Single emulsion skin care preparations, such as lotions and creams, of the oil-in-water type and water-in-oil type are well-known in the cosmetic art and are useful in the subject invention. Multiphase emulsion compositions, such as the water-in-oil-in-water type, are also useful in the subject invention. In general, such single or multiphase emulsions contain water, emollients, and emulsifiers as essential ingredients.

The compositions of the present invention can also be formulated into a solid formulation (e.g., a wax-based stick, soap bar composition, powder, bead, exfoliant, or a wipe containing liquid or powder).

The compositions of this invention can be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents. In some embodiments, such compositions include a combination of recombinant silk protein, water (Aqua), sodium C14-16 olefin sulfonate, glycerin, cocoa betaine, sodium benzoate, sodium hydroxide, calcium gluconate, sodium hyaluronate, propanediol, xanthan gum, gluconolactone, and tetrasodium glutamate diacetate. In some embodiments, compositions comprise a cleansing detergent, soap, serum, or toner. In a specific embodiment, the serum is aqueous-based. In another specific embodiment, the toner is alcohol-based.

The compositions useful in the present invention may be formulated as emulsions. If the composition is an emulsion, in some embodiments, from about 1% to about 10% or from about 2% to about 5% of the composition comprises an emulsifier. Emulsifiers may be nonionic, anionic or cationic. Suitable emulsifiers are disclosed in, for example, INCI Handbook, pp. 1673-1686. Lotions and creams can be formulated as emulsions. In some embodiments, the composition is an emulsion and the recombinant silk protein is an emulsifier. In some embodiments, the composition is an emulsion, the recombinant silk protein is an emulsifier, and the composition is free of other emulsifiers.

Yet another type of composition may be an ointment. An ointment may comprise a simple base of animal or vegetable oils or semi-solid hydrocarbons. An ointment may comprise from about 2% to about 10% of an emollient in addition to from about 0.1% to about 2% of a thickening agent. Examples of thickening agents include, e.g., cellulose derivatives (methyl cellulose and hydroxyl propylmethylcellulose), synthetic high molecular weight polymers (e.g., carboxyvinyl polymer and polyvinyl alcohol), plant hydrocolloids (e.g., karaya gum and tragacanth gum), clay thickeners (e.g., colloidal magnesium aluminum silicate and bentonite), carboxyvinyl polymers, carboxylic acid polymers, crosslinked polyacrylates, polyacrylamides, xanthan gum, and mixtures thereof.

The compositions useful in the subject invention may contain, in addition to the aforementioned components, a wide variety of additional oil-soluble materials and/or water-soluble materials conventionally used in compositions for use on skin, hair, and nails at their art-established levels.

The compositions of the present invention may be directly applied to the skin or may be applied onto other delivery implements such as wipes, sponges, brushes, and the like. The compositions may be used in products designed to be left on the skin, wiped from the skin, or rinsed off of the skin.

In some embodiments, the composition improves the appearance of skin, such as increasing skin firmness/plumpness, increasing elasticity, improving overall skin health, increasing hydration, accelerating and/or improving wound healing, improving pollution defense, reducing dermatological aging, decreasing skin fragility, preventing and reversing loss of collagen and/or elastin, preventing skin atrophy, promoting/accelerating cell turnover, increasing genetic expression, improving skin texture, preventing and decreasing fine lines and wrinkles, improving skin tone, enhancing skin thickness, decreasing pore size, minimizing skin discoloration, restoring skin luster, minimizing signs of fatigue, improving skin barrier function, minimizing skin dryness, preventing, reducing, or treating hyperpigmentation, improving the mitochondrial function of the skin, improves exfoliation, reduces toxicity, mattifying skin, reducing oxidative stress levels, attenuating pollution induced oxidative stress, attenuating UVA or UVB induced oxidative stress, or any combination thereof.

The compositions of various embodiments defend against pollutants and other irritants. As a result, many skin conditions, such as acne, the redness associated with rosacea (adult acne), and other inflammatory conditions can be actively managed by application of the cosmetic formulations.

### Coagulants

In some embodiments, a recombinant silk polypeptide containing composition and/or a silicone replacement component as described herein is exposed to a coagulant. This can change the properties of the composition/component to facilitate controlled aggregation of silk in the silk-based composition. In some embodiments, composition/component is submerged in a coagulant. In some embodiments, the composition/component is exposed to a coagulant mist or vapor. In one embodiment, an aqueous protein composition comprises or is submerged with or mixed with a coagulant. In some embodiments, a silk-based solid or semi-solid, such as a film, is submerged in or exposed to a vapor comprising coagulant. In some embodiments, methanol is used as an effective coagulant.

In some embodiments, alcohol (e.g., isopropanol, ethanol, or methanol) can be used as a coagulant or solvent. In some embodiments, 60%, 70%, 80%, 90% or 100% alcohol is used as a coagulant. In some embodiments, a salt can be used as a coagulant. Examples of salts include, but are not limited to, ammonium sulfate, sodium chloride, sodium sulfate, and other protein precipitating salts effective at a temperature from 20 to 60° C.

In some embodiments, a combination of one or more of water, acids, solvents, and salts, including, but not limited to, the following classes of chemicals of Brönsted-Lowry acids, Lewis acids, binary hydride acids, organic acids, metal cation acids, organic solvents, inorganic solvents, alkali metal salts, and alkaline earth metal salts can be used as a coagulant. In some embodiments, the acids comprise dilute hydrochloric acid, dilute sulfuric acid, formic acid, or acetic acid. In some embodiments, the solvents comprise ethanol, methanol, isopropanol, t-butyl alcohol, ethyl acetate, propylene glycol, or ethylene glycol. In some embodiments, the salts comprise LiCl, KCl, BeCl₂, MgCl₂, CaCl₂, NaCl, ZnCl₂, FeCl₃, ammonium sulfate, sodium sulfate, sodium acetate, or other salts of nitrates, sulfates or phosphates. In some embodiments, the coagulant is at a pH from 2.5 to 7.5.

### Other Additives

In some embodiments, a composition in accordance with the disclosure and/or the silicone replacement component thereof can include one or more additives. This can change the properties of the composition as it interacts with the skin. In some embodiments, the silk-based composition is submerged in the additive. In some embodiments, the composition/component is exposed to the additive mist or vapor. In one embodiment, an aqueous protein composition comprises or is submerged with or mixed with the additive. In some embodiments, a silk-based solid or semi-solid, such as a film, is submerged in or exposed to a vapor comprising the additive. In some embodiments, the silk-based gel is exposed to the additive prior to hallow powder formation (e.g., the silk-based gel and additive are co-spray dried together).

The additive can itself be inert or it can possess dermatological benefits of its own. The additive should also be physically and chemically compatible with the essential components described herein, and should not unduly impair stability, efficacy or other use benefits associated with the compositions of the present invention. The type of additive utilized in the present invention depends on the type of product form desired for the composition. In some embodiments, the additive is an acid textile dye.

Pigments are frequently added to cosmetic formulations to achieve a desired color for application to the skin. Such pigments are known and the concentrations required to achieve a desired coloring are readily determinable. Pigments may be inorganic or organic. Inorganic pigments include iron oxides (red, black, brown colors), manganese violet, ultramarines (green, blue, pink, red, or violet aluminum sulfosilicates), aquamarines, copper powder, mica, clays, silica, and titanium dioxide. Organic dyes that have been certified by the US FDA for cosmetic use generally have the prefix “D&C” and a suffix of a color and a number (for example, D&C Green #3).

Certain embodiments of the present invention contain from about 0% to about 30%, from about 1% to about 20%, from about 2% to about 15%, or from about 5% to about 15% of a colorant, on an anhydrous pigment weight basis. These are usually aluminum, barium or calcium salts or lakes. Dyes may be present at a concentration of from about 0% to about 3% and pearlizing agents and the like from 0% to about 10%. Such dyes in combination with recombinant silk proteins are stable and have a long shelf-life. The shelf-life of such compositions may be about 6 months, about 1 year, or about 2 years. In some embodiments, the shelf-life of such compositions may be at least 5 years.

There are no specific limitations as to the pigment, colorant, or filler powders used in the composition. Each may be a body pigment, inorganic white pigment, inorganic colored pigment, pearling agent, and the like. Specific examples are talc, mica, magnesium carbonate, calcium carbonate, magnesium silicate, aluminum magnesium silicate, silica, titanium dioxide, zinc oxide, red iron oxide, yellow iron oxide, black iron oxide, ultramarine, polyethylene powder, methacrylate powder, polystyrene powder, silk powder, crystalline cellulose, starch, titanated mica, iron oxide titanated mica, bismuth oxychloride, and the like.

Additional pigment/powder fillers include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; and inorganic white pigments such as magnesium oxide. Other useful powders are disclosed in U.S. Pat. No. 5,688,831, to El-Nokaly et al., issued Nov. 18, 1997, herein incorporated by reference in its entirety. These pigments and powders can be used independently or in combination.

Besides the silk protein, the composition according to the invention can further comprise a film-forming substance. Examples of film-forming substances include, e.g., cellulose derivatives, nitrocellulose, acrylic polymers or copolymers, acrylic, styrene, acrylate-styrene and vinyl resins, vinyl copolymers, polyester polymers, arylsulphonamide resins, and alkyde resins.

In some embodiments, the composition may include an amphoteric surfactant, a phospholipid, or a wax.

Examples of other additives include, but are not limited to, cannabidiol, foaming surfactants, depigmentation agents, reflectants, detangling/wet combing agents, amino acids and their derivatives, antimicrobial agents, allergy inhibitors, anti-acne agents, anti-aging agents, anti-wrinkling agents antiseptics, analgesics, antitussives, antipruritics, local anesthetics, anti-hair loss agents, hair growth promoting agents, hair growth inhibitor agents, antihistamines, antiinfectives, inflammation inhibitors, anti-emetics, anticholinergics, vasoconstrictors, vasodilators, wound healing promoters, peptides, polypeptides and proteins, deodorants and antiperspirants, medicament agents, skin emollients and skin moisturizers, skin firming agents, hair conditioners, hair softeners, hair moisturizers, vitamins, tanning agents, skin lightening agents, antifungals, depilating agents, shaving preparations, external analgesics, perfumes, counterirritants, hemorrhoidals, insecticides, poison ivy products, poison oak products, burn products, anti-diaper rash agents, prickly heat agents, make-up preparations, vitamins, herbal extracts, retinoids, flavenoids, sensates, anti-oxidants, skin conditioners, hair lighteners, chelating agents, cell turnover enhancers, sunscreens, anti-edema agents, collagen enhancers, and mixtures thereof.

Examples of suitable vitamins nonexclusively include vitamin B complex, including thiamine, nicotinic acid, biotin, pantothenic acid, choline, riboflavin, vitamin B6, vitamin B12, pyridoxine, inositol, camitine; vitamins A, C, D, E, K and their derivatives such as vitamin A palmitate and pro-vitamins, (e.g., panthenol (pro vitamin B5) and panthenol triacetate) and mixtures thereof.

Examples of sunscreen agents include, but are not limited to, avobenzone, benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and mixtures thereof.

The amount of additive to be combined with the composition may vary depending upon, for example, the ability of the additive to penetrate through the skin, hair, or nail; the specific additive chosen; the particular benefit desired; the sensitivity of the user to the additive; the health condition, age, and skin, hair, and/or nail condition of the user; and the like. In sum, the additive is used in a “safe and effective amount,” which is an amount that is high enough to deliver a desired skin, hair, or nail benefit or to modify a certain condition to be treated, but is low enough to avoid serious side effects, at a reasonable risk to benefit ratio within the scope of sound medical judgment.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

The compositions and methods of the present invention provide for skin equal or better performance for softness, quick absorption, easy spreadability (or “playtime”), lightweight film formation, and non-greasy afterfell as compared to compositions containing silicone elastomers. Additionally, if the skin is being treated with an SPF composition, then the invention provides equal or better performance for low white cast. The compositions and methods of the present invention provide for hair equal or better performance for long-lasting wear, shine, non-greasiness, frizz control, adding thickness to the hair, styling retention, electrostatic properties, resistance to heat, and UV-radiation and pollution defense.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended aspects.

In the aspects, articles such as “a,” “an,” and “the” may mean one or more than one unless indicated to the contrary or otherwise evident from the context. Aspects or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essential of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Recombinant Silk Polypeptide as Replacement for Silicones in SPF Formulation

In this example, we show that recombinant silk polypeptide matches and outperforms silicone (i.e., blend of linear silicones and cyclosiloxanes) for certain characteristics of a SPF skincare product. A mineral SPF formulation that originally contained 2% combined linear silicones and cyclosiloxanes (1% of each component) was reformulated with 1.5% recombinant silk polypeptide and evaluated for its aesthetic profile. The recombinant silk polypeptide was preferred over the silicone formulation in a blind comparison for white cast, absorption, film feel, and oiliness, while matching the silicone formulation in playtime and softness performance.

Methods: The SPF formulation was prepared similar to standard practices in the field. The water phase ingredients were mixed together separately, and the oil phase ingredients were mixed together separately. The two were combined with gentle mixing at an elevated temperature of 80-85 degree C. The ingredients were mixed continually until uniform and then cooled to 30-45 degree C. An 18B recombinant silk polypeptide comprising SEQ ID NO: 2878 was added to the formulation after the water and oil phases were mixed together and cooled to 30-45 degree C. The formulation was continually mixed while cooling to room temperature. Water was used to make up the difference in weight percent of recombinant silk polypeptide and silicone elastomer between formulations. For this formulation, a recombinant silk polypeptide in the powder form was used.

The spider chart was prepared with a blind comparison test, where subjects were asked to compare the two formulations on a scale of 1-5 for six different attributes (Key: 1=Unfavorable, 2=Bad, 3=Satisfactory, 4=Good, 5=Excellent). Ten (10) different individuals tested the products on their face for one full week. Skin types varied from oily, dry, and combination.

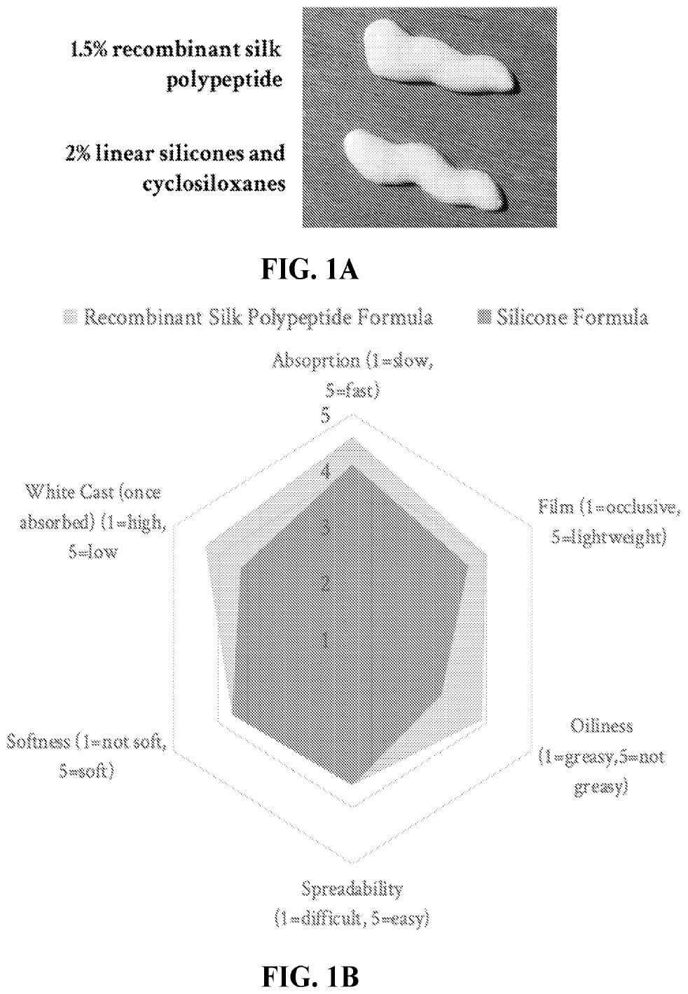

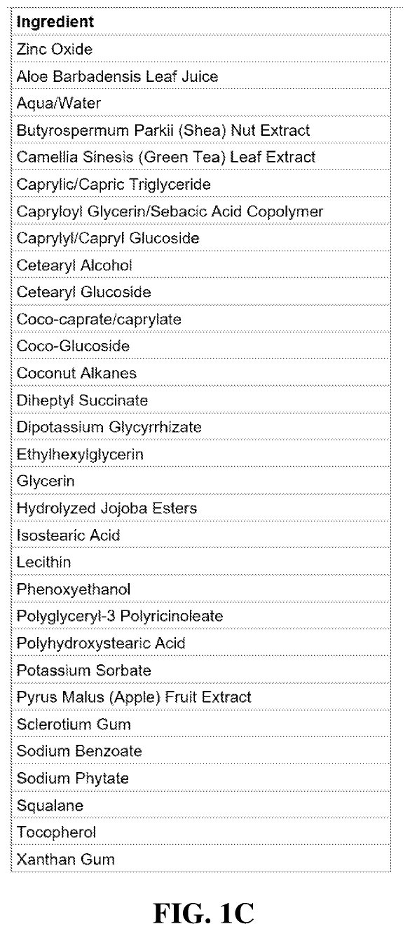

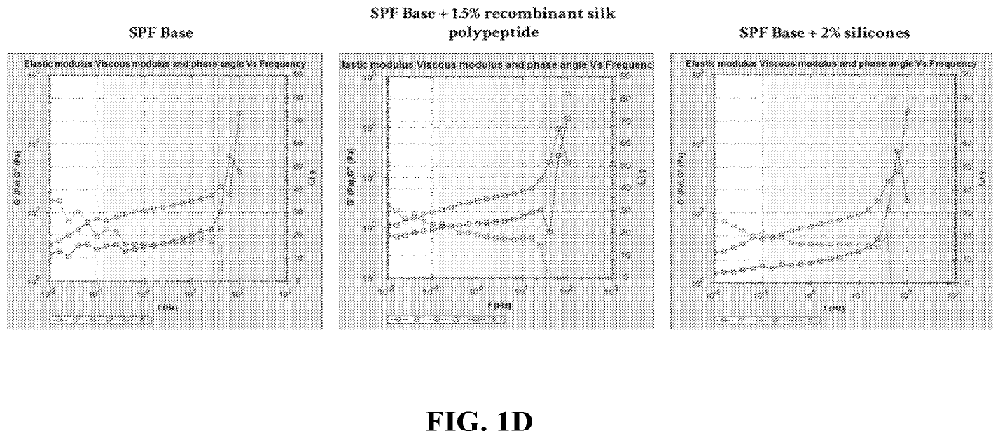

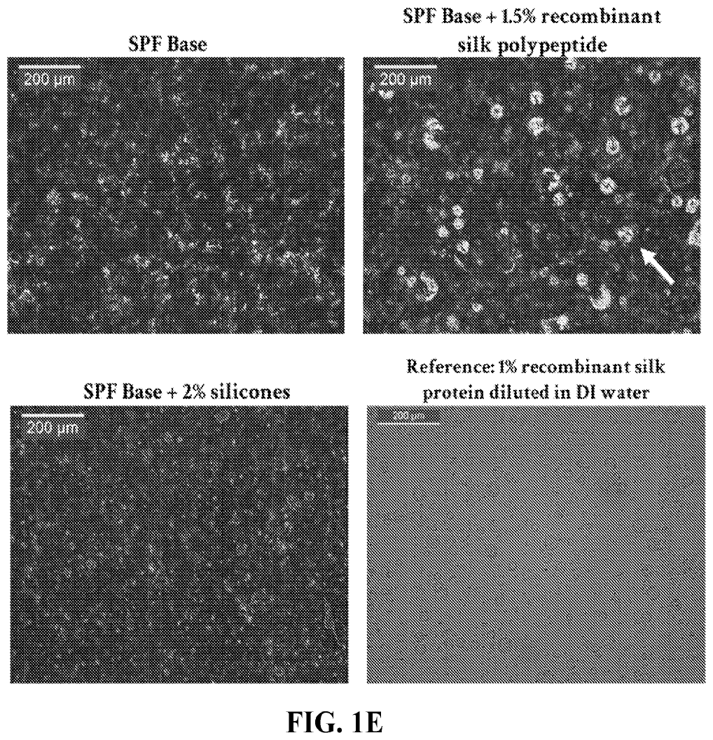

FIG. 1A illustrates a top-down view of the SPF formulations

FIG. 1B shows a spider chart plotting the data from the blind test comparison of 10 subjects. The 1.5% recombinant silk polypeptide formulation outperformed the 2% silicone formulation for desirable white cast, absorption, film feel, and oiliness. The 1.5% recombinant silk polypeptide formulation performed similarly as the 2% silicone formulation for spreadability and softness.

FIG. 1C: Table of ingredients used in the SPF formulations (not including the recombinant silk polypeptide and silicones).

FIG. 1D: While the SPF formulation that contains the 1.5% recombinant silk polypeptide outperformed the 2% silicone formulation for desirable white cast, absorption, film feel, and oiliness, the rheology data shows similarity between the three formulations. The frequency sweeps show similar magnitude and shape of the G′ and G″ curve. This data supports that the SPF formulations have similar gel-like structural properties.

FIG. 1E: Light microscopy images of SPF base and SPF base formulated with 1% recombinant silk polypeptide protein or 5% silicone elastomer ingredient. The SPF base and the 5% silicone elastomer samples exhibit typical dispersion morphology, which has regions that are more transparent and other regions that are opaque. The 1% recombinant silk polypeptide sample clearly has the visible presence of particles that are hollow and 2-200 μm in diameter and show up as bright circles. A white arrow is added to the figure to point out an example silk powder particle. A reference image of 1% recombinant silk polypeptide powder suspended in water is included.

### Example 2: Recombinant Silk Polypeptide as Replacement for Silicones in Color Cosmetic Product

Recombinant silk polypeptide matches and outperforms silicone elastomer for certain characteristics of a color cosmetic product. A pigmented 3-in-1 cream eye/cheek/lip formulation was prepared with either 1% recombinant silk polypeptide or 5% or 10% silicone elastomer and evaluated for its aesthetic profile. The recombinant silk polypeptide formulation outperformed the silicone elastomer and provided improved spreadability, even pigment delivery, and soft dry-down (i.e., not sticky or greasy like the silicone elastomer version) that was also resistant to wipe-off.

Methods: The color cosmetic formulation was prepared similar to standard practices in the field. All waxes, oils, and solvents were brought to room temperature or to an elevated temperature separately, for example, to greater than 37 degrees C. but less than 90 degrees C. The oils and solvents were mixed with the pigment and lightly milled. Then the waxes were added to the mixture and cooled. The b-silk protein (i.e., 18B recombinant silk polypeptide comprising SEQ ID NO: 2878) and/or silicone elastomer ingredients can be added at any stage of the process. Water was used to make up the difference in weight percent of recombinant silk polypeptide and silicone elastomer between formulations. For this formulation, a recombinant silk polypeptide in powder form was used, and a silicone elastomer ingredient with approximately 30% solids was used.

Testing of the pigment application and wipe off include applying 10 mg of the product to a 1 cm×1 cm square area and allowing the product to dry for 15 minutes. To test wipe off, a white tissue paper was laid on the skin and 200 g mass was set on the skin. The tissue was pulled over the skin 3 times and the wipe off characteristic was evaluated visually.

Rheology measurements were taken with a Kinexus Lab+Rheometer using a 2 degree/20 mm cone and plate geometry. To measure the G′ and G″ of the materials a frequency sweep was performed at 0.1% strain rate. The start frequency was 100 s⁻¹ and the end frequency was 0.01 s⁻¹.

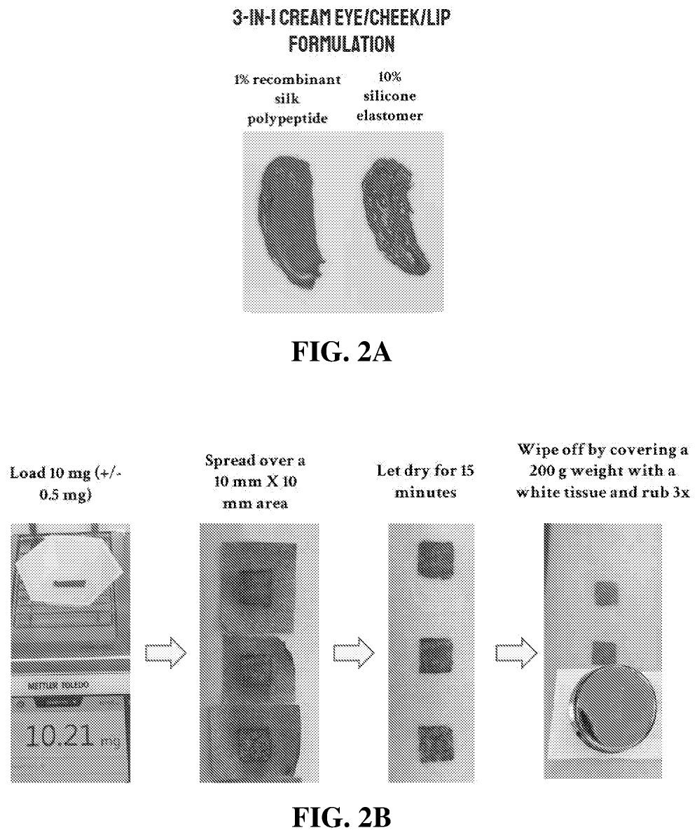

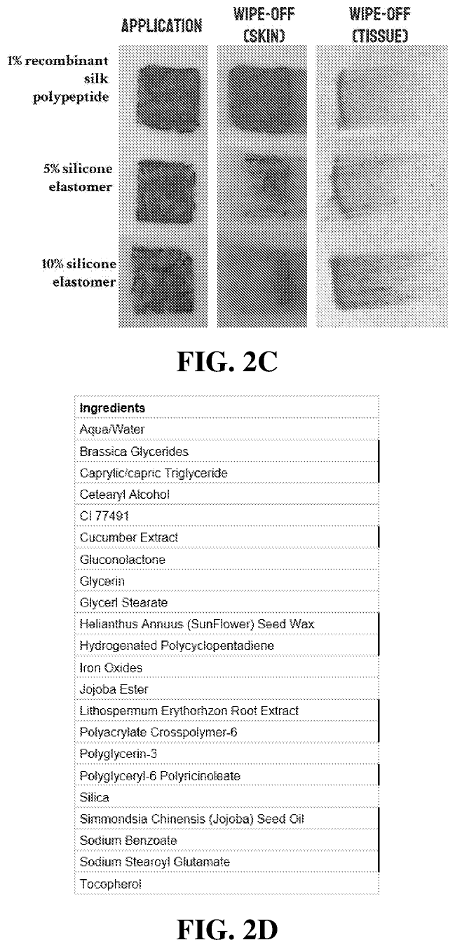

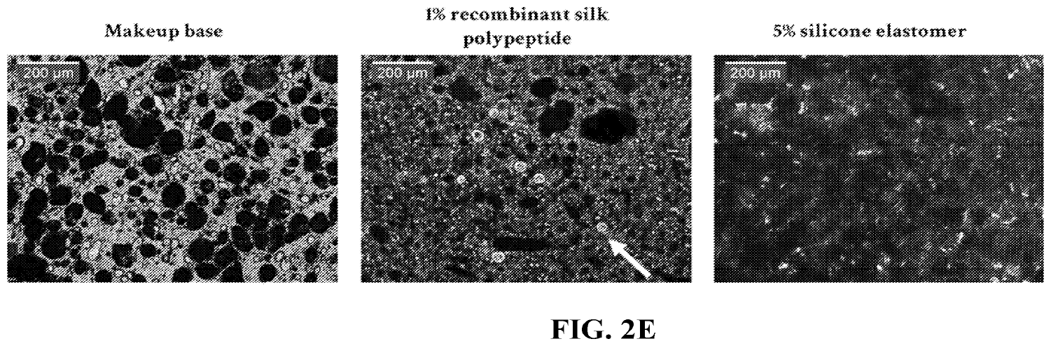

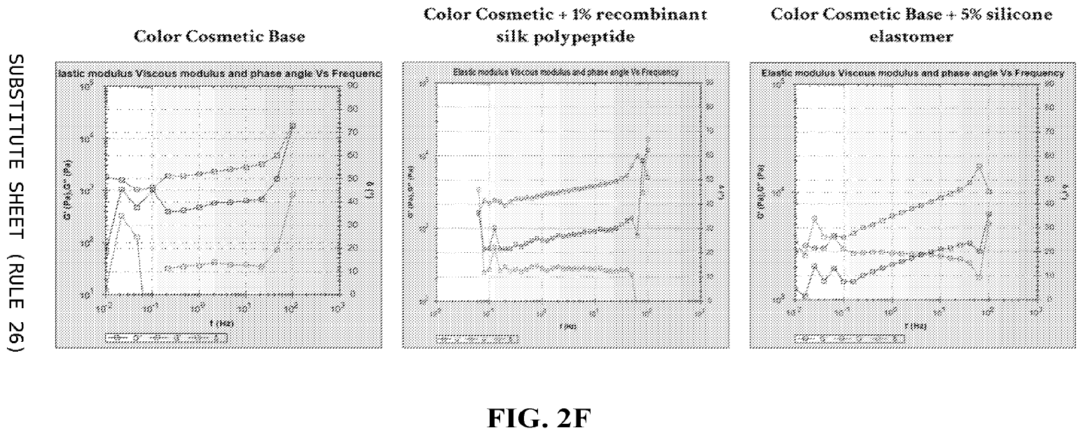

FIG. 2A: Top-down view of the color cosmetic formulations.

FIG. 2B: Step-by-step visual of how the color cosmetic was evaluated for pigment delivery and substantiation to skin.

FIG. 2C: Representative images of color application and wipe-off of the product. Compared to the 5% and 10% elastomer products, the recombinant silk polypeptide had more even pigment spreading on skin and was more substantial to skin during wipe-off.

FIG. 2D: Table of ingredients used in the color cosmetic formulations (not including the recombinant silk polypeptide and silicones).

FIG. 2E: Light microscopy images of color cosmetic base and the base formulated with 1% b-silk protein or 5% silicone elastomer ingredient. The SPF base and the 5% silicone elastomer samples exhibit typical dispersion morphology, which has regions that are more transparent and other regions that are opaque. The 1% recombinant silk polypeptide sample clearly has the visible presence of particles that are hollow and 2-200 μm in diameter and show up as bright circles. A white arrow is added to the figure to point out an example silk powder particle.

FIG. 2F: While the color cosmetic formulation that contains the 1% recombinant silk protein is distinctly different from the 5% and 10% formulations as measured by performance characteristics and microscopy, the rheology data shows similarity between the three formulations. The frequency sweeps show similar magnitude and shape of the G′ and G″ curve. This data indicates that the color cosmetics have similar gel-like structural properties.

### Example 3: Recombinant Silk Polypeptide as Replacement for Silicone Elastomers in a Hair Serum

A leave-in hair serum was prepared with either 1% recombinant silk polypeptide or 5% silicone elastomer. The formulations behaved similarly for styling including shine, curl retention, and frizz control.

Methods: The hair serum formulation was prepared similar to standard practices in the field. With moderate mixing, all the ingredients were combined until completely uniform. Water was used to make up the difference in weight percent of recombinant silk polypeptide (i.e., 18B silk) and silicone elastomer between formulations. For this formulation, a recombinant silk polypeptide in the powder form was used and a silicone elastomer ingredient with approximately 30% solids was used.

Rheology measurements were taken with a Kinexus Lab+Rheometer using a 2 degree/20 mm cone and plate geometry. To measure the G′ and G″ of the materials an amplitude sweep was performed at 1 s⁻¹ frequency over a range of shear strains from 0.01% to 250%. Additionally a frequency sweep was performed at 0.1% strain rate. The start frequency was 100 s⁻¹ and the end frequency was 0.01 s⁻¹.

To evaluate the morphology of the formulation on the hair, 0.25 g of the formulation was evenly spread onto 0.35 g of yak hair with gentle massaging. The hair was then dried with hot air at 200 degrees C. for 5 minutes until dry to the touch. Scanning electron microscopy (SEM) used a focus electron beam to assess the morphology of materials through the secondary electrons. The electron beam was scanned in a raster pattern to collect micrographs at scales between 1 mm and 10 nm or between 10× and 100,000× magnification. The SEM method used low vacuum (1 to 10 torr), avoiding the need for dehydrating or sputter coating biological samples.

Light microscopy images were used to examine the formulation powder morphology and were obtained using a Leica DM750P light microscope, using a 10× objective. The Microscope was coupled to the complementary PC based image analysis Leica Application Suite, LAS V4.9 for capturing images.

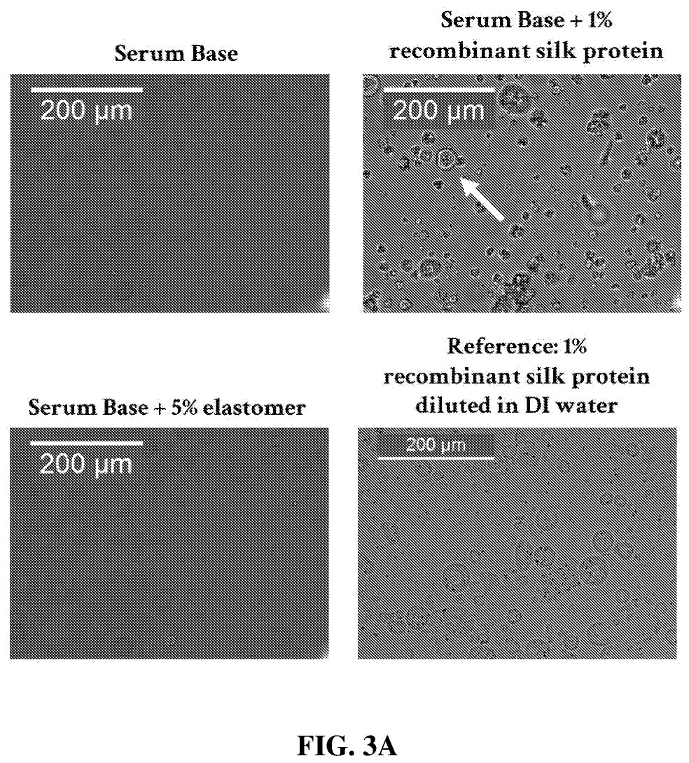

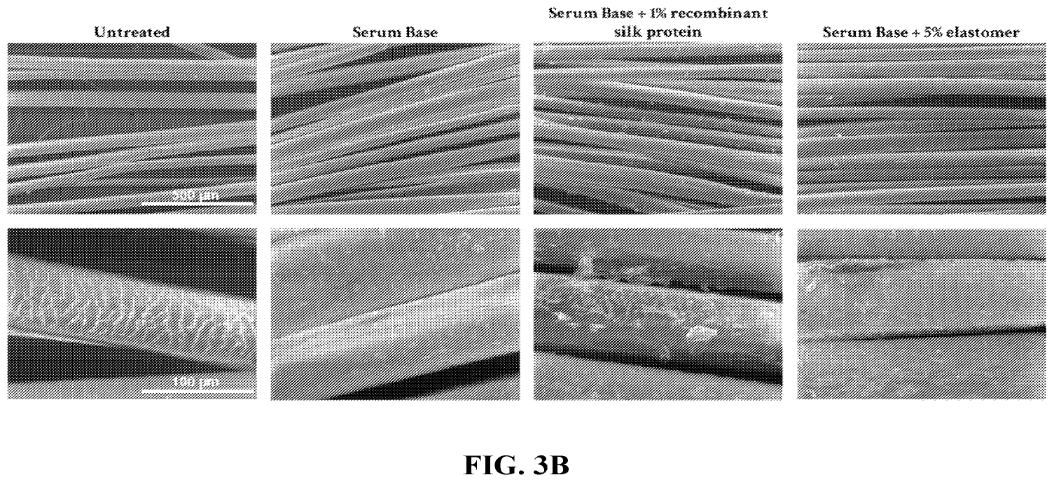

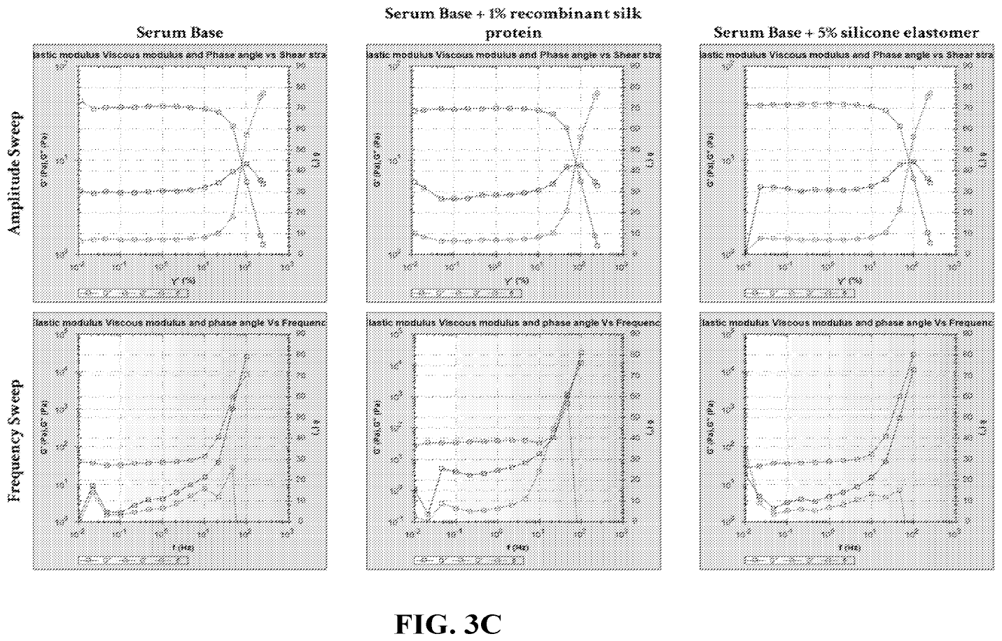

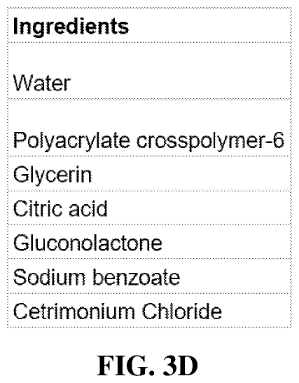

FIG. 3A: Light microscopy images of hair serum formulations with and without 1% recombinant silk polypeptide and 5% silicone elastomer. The base serum and the silicone elastomer samples have uniform structure as evidenced by the absence of any visually distinct features. The 1% recombinant silk polypeptide sample clearly has the visible presence of recombinant silk polypeptide particles that are hollow and 2-200 μm in diameter.

FIG. 3B: SEM images of the hair serum formulation dispersed onto yak hair.

Approximately 0.25 g of serum was dispersed onto 0.35 g of hair. Compared to the untreated, all serum samples visibly coat the hair strand. Only the recombinant silk polypeptide serum resulted in a distinct surface morphology that is marked by regions of smooth film, regions of rough film, and regions of particles.

FIG. 3C: While the serum formulation that contained the 1% recombinant silk protein was distinctly different as measured by microscopy, the rheology data showed similarity between the three formulations. The amplitude sweeps show similar magnitude and lengths of the linear viscoelastic regions (refer to the G′ curve). Additionally, the frequency sweeps show similar magnitude and length of the G′ curve. This data indicates that the hair serums have similar gel-like structural properties and will perform rheologically similarly.

FIG. 3D: Table of ingredients used in the hair serum formulations (not including the recombinant silk polypeptide and silicones).

### Example 4: Rheological Comparison and Morphological Comparison of the Recombinant Silk Polypeptide to the Silicone Elastomer Ingredient

Methods: Rheology measurements were taken with a Kinexus Lab+Rheometer using a 2 degree/20 mm cone and plate geometry. To measure the G′ and G″ of the materials, a frequency sweep was performed at 0.1% strain rate. The start frequency was 100 s⁻¹ and the end frequency was 0.01 s⁻¹. Three samples were taken per decade. Viscosity measurements were taken by shearing the sample over the shear rate of 0.1 s⁻¹-1000 s⁻¹. All rheology and viscosity measurements were taken at 22-27 degrees. The recombinant silk protein solution (comprising 18B silk) was prepared by mixing powder with DI water in a high shear mixer.

To evaluate the morphology of the formulation on the hair, 0.25 g of the formulation was evenly spread onto 0.35 g of yak hair with gentle massaging. The hair was then dried with hot air at 200 degrees C. for 5 minutes until dry to the touch. Scanning electron microscopy (SEM) used a focus electron beam to assess the morphology of materials through the secondary electrons. The electron beam was scanned in a raster pattern to collect micrographs at scales between 1 mm and 10 nm or between 10× and 100,000× magnification. The SEM method used low vacuum (1 to 10 torr), avoiding the need for dehydrating or sputter coating biological samples.

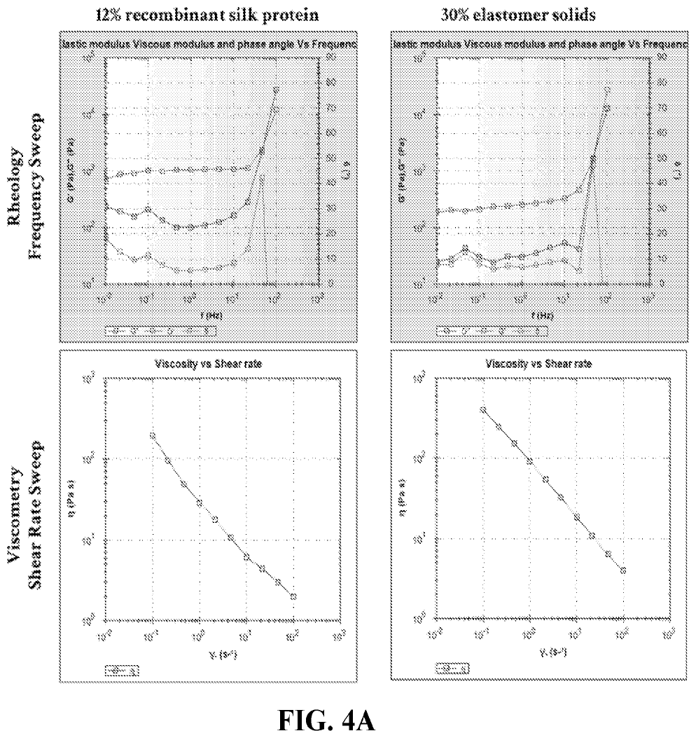

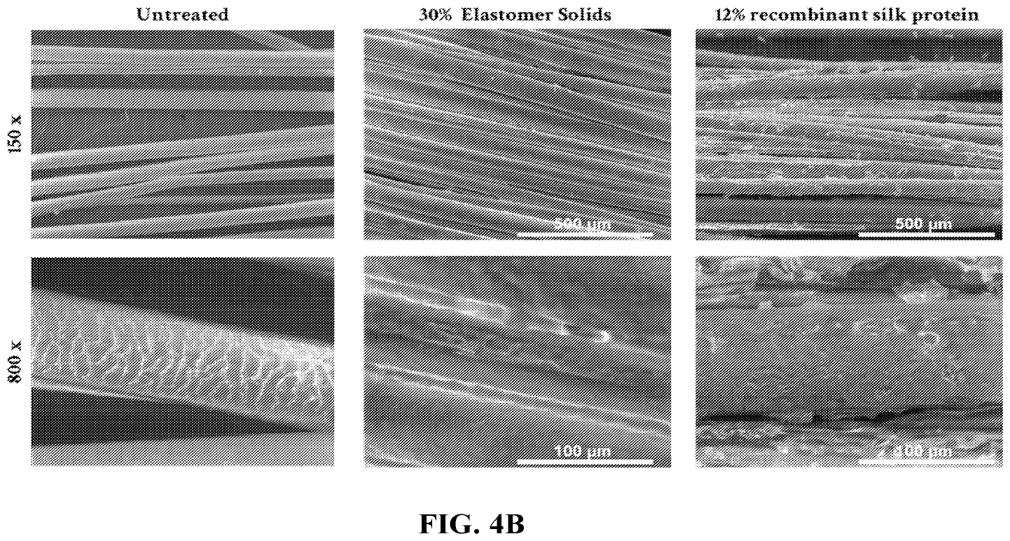

FIG. 4A: Comparison of the G′ and G″ of an industry standard silicone elastomer gel (dry solids of 30%) and 12% recombinant silk polypeptide. Despite silicone elastomers and recombinant silk polypeptides having very different molecular make ups, crosslinking chemistries, and macromolecular morphologies, both materials exhibit similar rheological properties. This is typified by a flat G′ curve in the rheological frequency sweep. This flat curve indicates that both materials are structured gels. In the viscosity curves, both materials display similar non-Newtonian, shear thinning profiles typical of polymeric solutions and suspensions.

FIG. 4B: SEM images of the neat recombinant silk polypeptide and silicone elastomer dispersed onto yak hair. Approximately 0.25 g of serum was dispersed onto 0.35 g of hair. Compared to the untreated, both the recombinant silk polypeptide and silicone elastomer deposited a visible film on the hair shaft. The silicone elastomer film is relatively smooth. The recombinant silk polypeptide film is characterized by a more rough surface with visible pits and particles protruding from its surface.

Viscosity measurements as set forth in this Example 4 also were obtained for commercially available silicone elastomers in diluents and 18B recombinant silk polypeptide in water as follows: 1) Specsil K-13 (Innospec, Englewood, Colorado), 10% w/w in cyclopentasiloxane; 2) Silmer G-162-F5 (Siltech, Toronto, Ontario), 15% w/w in dimethicone; 3) CHT-beausil gel 8055 (CHT, Tubingen, Germany), 5% w/w in dimethicone; 4) Dowsil HMW 2220 non-ionic emulsion (Dow, Midland, Michigan), 60% w/w in C12-13 Pareth-23 and C12-13 Pareth 3; and 5) 18B recombinant silk polypeptide (19% recombinant silk polypeptide in water; 22% recombinant silk polypeptide in water; 25% silk polypeptide in water; and 27% silk polypeptide in water). All materials tested displayed similar non-Newtonian, shear thinning profiles typical of polymeric solutions and suspensions.

Comparison of the G′ and G″ of CHT-beausil gel 8055, 5% w/w in dimethicone, with 27% 18B recombinant silk polypeptide in water also was performed. Both materials exhibited similar curves where G′ dominated over G″ in the magnitude range of 30 Pa up to 16000 Pa over the angular frequency range of 1-100 Hz.

### Example 5: Style Retention Test of a Leave-In Hair Styling Serum Containing Recombinant Silk Polypeptide

Hair swatches of virgin hair (medium brown, 3 g, 1 in width, 8 in length, sealed with hot melt) were treated with 1 g of leave-in hair serum. The leave-in hair serum contained serum base only, or serum base with either 1% silk polypeptide, 1% keratin ingredient, or 5% silicone elastomer ingredient. The formulation for the serum base is outlined in FIG. 3D. The hair swatches were rolled up tightly into a ¾″ diameter curler and mounted to a grid board with 1 cm wide markings. The samples were incubated in an oven at 50° C., 70% humidity for eight (8) hours. After 8 hours, samples were removed from the oven, cooled to room temperature, and unrolled.

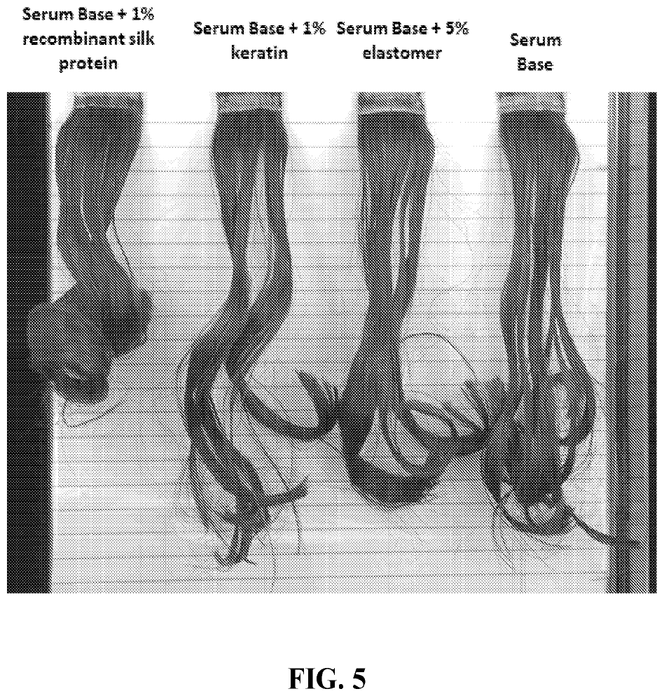

FIG. 5 shows image of hair swatches after exposure to the curl retention testing modality as described above. The recombinant silk polypeptide sample outperformed all other samples with improved curl retention. Quantification of the hair swatch length after exposure to the curl retention testing modality is shown in Table 2. The recombinant silk polypeptide exhibited 35% more curl retention than the base serum and 28% more curl retention than the silicone elastomer sample.

TABLE 2

| | Length | % improvement over |
|---|---|---|
| Sample | [cm] | serum base |
| Serum Base | 18 | 0 |
| Serum Base + 5% | 16 | 11 |
| elastomer | | |
| Serum Base + 1% | 18.5 | −6 |
| keratin | | |
| Serum Base + 1% | 11.5 | 35 |
| recombinant silk | | |
| protein | | |

### Example 6: Wash-Off Shampoo Formulation

A wash-off shampoo formulation was developed that contained a range of silk polypeptide (0.05%, 0.5%, and 1%). These formulas were compared to a placebo—a formulation in which the silk polypeptide was removed and the difference was covered by adding more water. A silicone elastomer version was also made—the silk polypeptide was omitted and replaced with silicone elastomer at 3% loading level (the difference in mass was compensated for by adding less water).

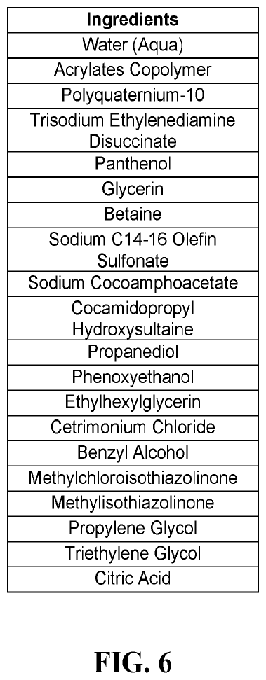

The shampoo formulation was prepared similar to standard practices outlined in Example 3. For this formulation, a recombinant silk polypeptide in powder form was used, and a silicone elastomer ingredient with approximately 60% solids was used. A list of the ingredients in the formulation (not including the silk polypeptide or silicone elastomer) with a range of loading levels is provided in FIG. 6.

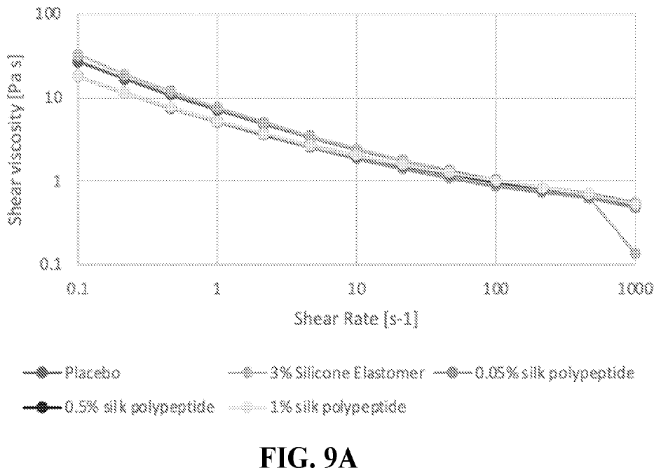

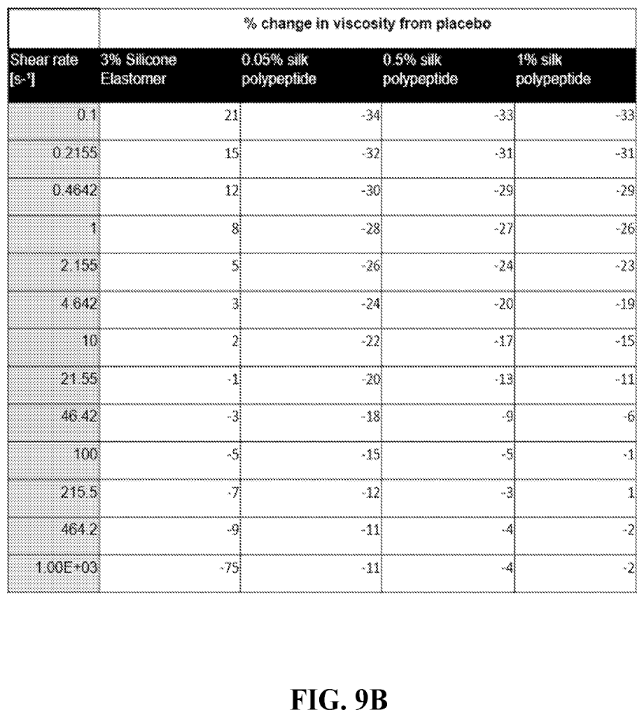

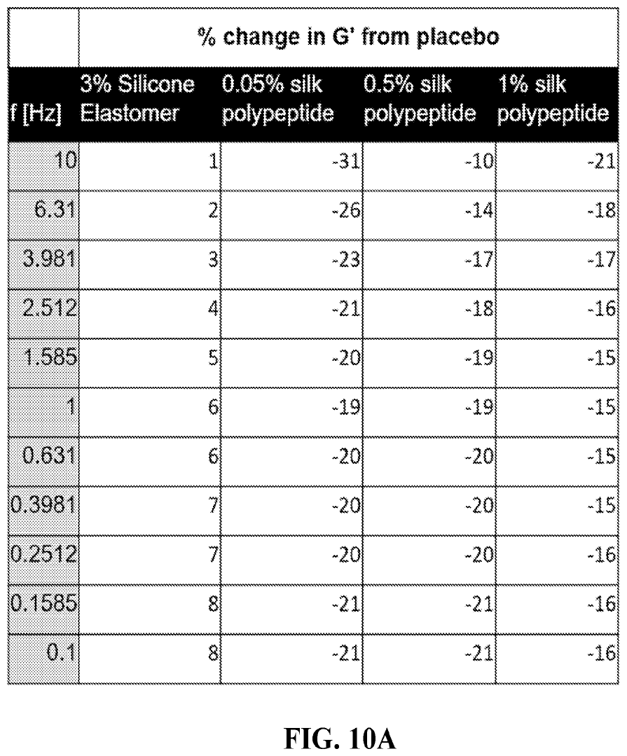

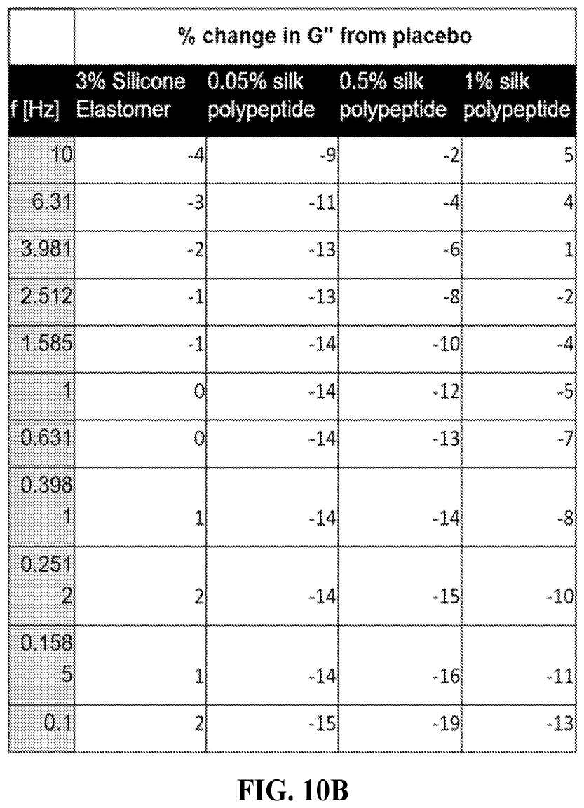

Viscosity and Rheology was evaluated using the method in Example 3. The viscosity curves of the formulations demonstrated that all formulations displayed a similar shear thinning behavior. FIGS. 9A and 9B demonstrated that over the range of 1-1000 s⁻¹ shear rates, the silk polypeptide samples did not deviate from the placebo by more than 34%. Rheology curves of the formulations demonstrated that, in all cases, the formulations exhibited similar shapes and the G′ dominates over the G″. FIGS. 10A and 10B include a table of the rheology curve data points demonstrated that the over the range of angular frequencies of 0.1 Hz to 10 Hz, the silk polypeptide never deviated from the placebo by more than 31% for G′ and 19% for G″. Inclusion of silk polypeptide as described herein did not significantly change rheology profile compared to the shampoo placebo.

Light microscopy was evaluated using the method in Example 3. The silk polypeptide can be visually detected at 0.05% loading level.

For split head evaluation, each side of the head was wetted and two grams of the shampoo sample was massaged into the hair for about 45 seconds. Hair was rinsed for 3 minutes. Hair was combed and then blow dried for 5-10 minutes. The 0.05% silk polypeptide loading level performed similarly to the silicone elastomer sample in terms of style control. As the silk polypeptide concentration increased, the silk polypeptide sample outperformed silicone elastomers for style control.

SEM images of how the various products deposit on the hair shaft were obtained. Approximately 0.25 g of shampoo was dispersed onto 0.35 g of hair and massaged for about 45 seconds. Hair was then rinsed with DI water for 45 seconds. Hair swatches were dried at ambient temperature/humidity for 24 hours. Samples were evaluated with SEM using the method in Example 3. No significant differences were detected in how the material (silk protein versus silicone elastomer formulation) affects the surface of the hair shaft.

A list of the ingredients in the formulation (not including the silk polypeptide or silicone elastomer) with a range of loading levels.

### Example 7: Leave-on Skin Serum

A leave-on skin serum formulation was developed that contained a range of silk polypeptide (0.1%, 0.25%, 0.5% and 0.75%). These formulas were compared to a placebo—a formulation in which the silk polypeptide was removed and the difference was covered by adding more water. A silicone elastomer version was also made—the silk polypeptide was omitted and replaced with silicone elastomer at 0.75% loading level (the difference in mass was compensated for by adding less water).

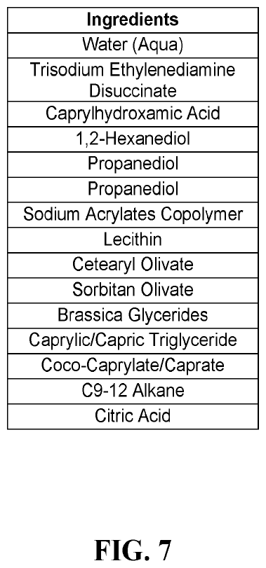

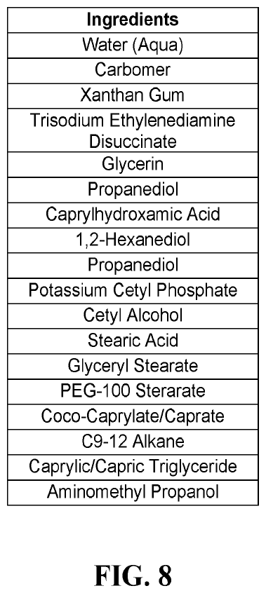

The skin serum formulation was prepared similar to standard practices outlined in Example 1. For this formulation, a recombinant silk polypeptide in the powder form was used and a silicone elastomer ingredient with approximately 15% solids was used. A list of the ingredients in the formulation (not including the silk polypeptide or silicone elastomer) with a range of loading levels is provided in FIG. 7.

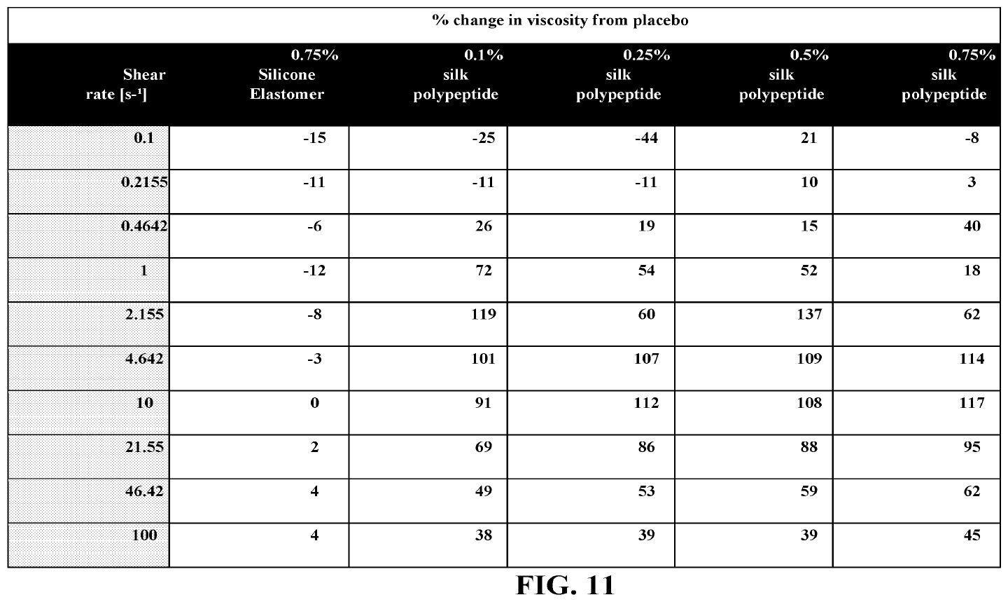

Viscosity and Rheology was evaluated using the method in Example 3. Viscosity curves of the formulations demonstrated that all formulations had a similar shear thinning behavior. FIG. 11 is a table of the viscosity curve data points showed that over the range of 1-100 s⁻¹ shear rates, the silk polypeptide samples deviate from the placebo by up to 137%, representing a significant and beneficial increase in viscosity. However the shape of the G′ and G″ curve was observed to remain substantially similar between the compositions of the disclosure and the placebo.

Light microscopy was evaluated using the method in Example 3. The silk polypeptide can be visually detected at 0.1% loading level.

### Example 8: Leave-on Skin Primer

A leave-on skin primer formulation was developed that contained 2% silk polypeptide. The formula was compared to a placebo—a formulation in which the silk polypeptide was removed and the difference was covered by adding more water. A silicone elastomer version was also made—the silk polypeptide was omitted and replaced with silicone elastomer at 7.5% loading level (the difference in mass was compensated for by adding less caprylic/capric triglyceride).

The skin primer formulation was prepared similar to standard practices outlined in Example 1. For this formulation a recombinant silk polypeptide in the powder form was used, and a silicone elastomer ingredient with approximately 15% solids was used.

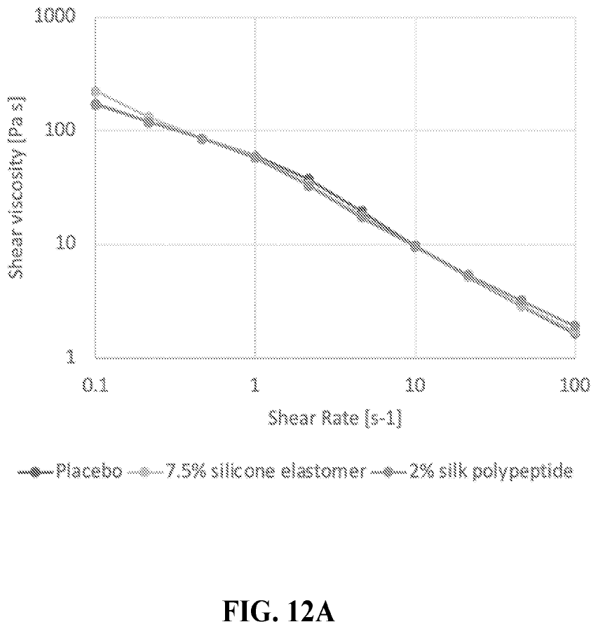

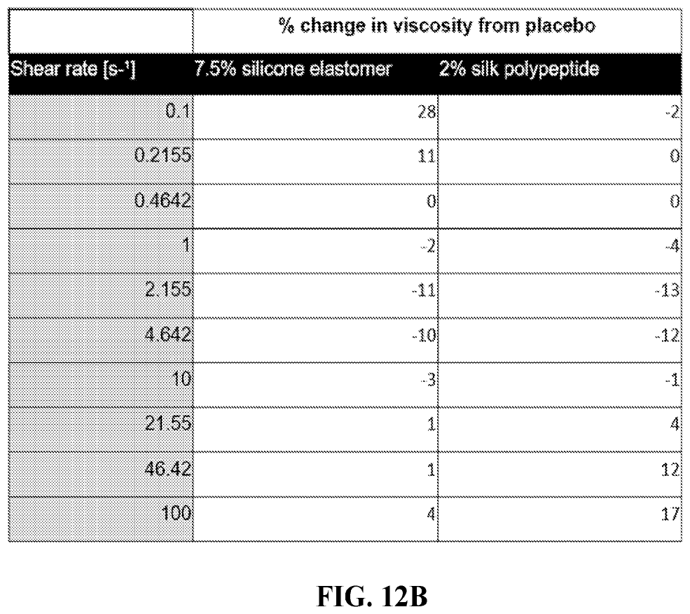

Referring to FIGS. 12A and 12B, a the viscosity curve data points showed that over the range of 1-100 s⁻¹ shear rates, the silk polypeptide samples did not deviate from the placebo by more than 35%.

### OTHER EMBODIMENTS

It is to be understood that the words which have been used are words of description rather than limitation, and that changes may be made within the purview of the appended aspects without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended aspects so as to provide the broadest possible interpretation of such aspects in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### ASPECTS

1. A composition comprising recombinant silk polypeptide and a solvent.
2. The composition of aspect 1, wherein said composition is a cosmetic or skincare product.
3. The composition of any one of the above aspects, wherein said composition comprises less than 30%, less than 20%, less than 10%, less than 5%, less than 4%, less than 3%, less than 2%, less than 1%, less than 0.5%, less than 0.1%, less than 0.05%, less than 0.01%, less than 0.005%, or less than 0.001% silicone elastomer.
4. The composition of any one of the above aspects, wherein said composition does not comprise a silicone elastomer.
5. The composition of any one of the above aspects, wherein said composition comprises at least 0.1%, at least 0.5%, at least 1%, at least 2%, at least 5%, at least 10%, at least 15%, at least 20%, at least 30%, at least 40%, or at least 50% recombinant silk polypeptide.
6. The composition of any one of the above aspects wherein said composition is a cosmetic or skincare product.
7. The composition of any one of the above aspects, wherein the recombinant silk polypeptide is greater than 100 amino acids in length.
8. The composition of any one of the above aspects, wherein the recombinant silk polypeptide is self-assembled into a semicrystalline state.
9. The composition of aspect 8, wherein the crystalline portion of said recombinant silk polypeptide is characterized by beta-sheet crosslinks.
10. The composition of any one of the above aspects, wherein said recombinant silk has poor solubility in a solvent selected from the group consisting of: water at a pH from 3 to 8, other polar and non polar solvents (for example, hexanol, hexane, benzene), oils, waxes, surfactants (for example, anionic, non-ionic, cationic, amphoteric).
11. The composition of any one of the above aspects, wherein said recombinant silk forms a heterogeneous dispersion in a solvent selected from the group consisting of: water at a pH from 3 to 8, other polar and non polar solvents (hexanol, hexane, benzene), oils, waxes, surfactants (anionic, non-ionic, cationic, amphoteric).
12. The composition of any one of aspects 1-7, wherein said recombinant silk polypeptide forms part of a randomly structure gel.
13. The composition of aspect 12, wherein said gel comprises preservative agents or chelating agents.
14. The composition of any one of aspects 1-7, wherein said recombinant silk polypeptide forms part of a hollow powder.
15. The composition of aspect 12, wherein said powder comprises a preservative agent or a chelating agent.
16. The composition of any one of aspects 14 and 15, wherein said hollow powder is suspended in a solvent.
17. The composition of aspect 16, wherein said solvent is selected from the group consisting of: an aqueous solvent, a polar solvent, a non-polar solvent, an oil solvent, a wax solvent, or a surfactant.
18. The composition of any one of aspects 16 and 17, wherein said solvent comprises a preservative agent or a chelating agent.
19. The composition of any one of the above aspects, wherein said composition comprises from 0.01 to 0.5% recombinant silk polypeptide, from 0.05% to 5% recombinant silk polypeptide, from 0.5% to 5% recombinant silk polypeptide, from 1% to 5% recombinant silk polypeptide, or from 5% to 20% recombinant silk polypeptide.
20. The composition of any one of the above aspects, wherein said recombinant silk polypeptide replaces a silicone elastomer traditionally used with said composition.
21. The composition of aspect 20, wherein said recombinant silk polypeptide replaces said silicone elastomer at a silicon elastomer: silk polypeptide ratio from 1:1 to 10:1.
22. The composition of any one of the above aspects, wherein said composition comprises a performance characteristic selected from the group consisting of: silky, smooth, and powdery feel; decrease in glossiness on the skin; enhanced shine on the hair; vibrant and efficient pigment delivery; easy spreadability; quick absorption time; mattification; wrinkle blurring effect; hair style retention; heat resistance for hair; UV and pollution defense; and increased viscosity.
23. The composition of any one of the above aspects, wherein said recombinant silk polypeptide comprises SEQ ID NO: 2878.
24 The composition of any one of the above aspects, wherein said composition is an SPF formulation, a color cosmetic, or a hair serum.
25. The composition of any one of aspects 1-24, wherein said composition comprises water.
26. The composition of any one of aspects 1-24, wherein said composition comprises water, glycerin, and sodium benzoate.
27. The composition of any one of aspects 1-24, wherein said composition comprises: Zinc Oxide, Aloe Barbadensis Leaf Juice, Water, Butyrospermum Parkii (Shea) Nut Extract, Camellia Sinesis (Green Tea) Leaf Extract, Caprylic/Capric Triglyceride, Capryloyl Glycerin/Sebacic Acid Copolymer, Caprylyl/Capryl Glucoside, Cetearyl Alcohol, Cetearyl Glucoside, Coco-caprate/caprylate, Coco-Glucoside, Coconut Alkanes, Diheptyl Succinate, Dipotassium Glycyrrhizate, Ethylhexylglycerin, Glycerin, Hydrolyzed Jojoba Esters, Isostearic Acid, Lecithin, Phenoxyethanol, Polyglyceryl-3 Polyricinoleate, Polyhydroxystearic Acid, Potassium Sorbate, Pyrus Malus (Apple) Fruit Extract, Sclerotium Gum, Sodium Benzoate, Sodium Phytate, Squalane, Tocopherol, Xanthan Gum, or any combination thereof.
28. The composition of any one of aspects 1-24, wherein said composition comprises: water, brassica glycerides, caprylic/capric triglyceride, cetearyl alcohol, CI 77491, cucumber extract, gluconolactone, glycerin, glycerl stearate, Helianthus annuus (sunflower) seed wax, hydrogenated polycyclopentadiene, iron oxides, jojoba ester, lithospermum erythorhzon root extract, plyacrylate crosspolymer-y, polyglycerin-3, polyglyceryl-6 polyricinoleate, silica, Simmondsia chinensis (jojoba) seed oil, sodium benzoate, sodium stearoyl glutamate, tocopherol, or any combination thereof.
29. The composition of any one of aspects 1-24, wherein said composition comprises: Water, Polyacrylate crosspolymer-6, Glycerin, Citric acid, Gluconolactone, Sodium benzoate, Cetrimonium Chloride, or any combination thereof.
30. The composition of any one of the above aspects, wherein the solvent comprises an aqueous solvent, an alcohol, an oil-based solvent, or a silicone.
31. The composition of any one of the above aspects, wherein the solvent is selected from the group consisting of: water, glycerin, deionized water, olive oil, and pentylene glycol.
32. The composition of any one of the above aspects, wherein the composition cleanses the skin.
33. The composition of any one of the above aspects, wherein the composition further comprises a dye.

What is claimed is:

1. A cosmetic, skin, or hair care composition having a silicone replacement, comprising:
one or more active ingredients for cosmetic, skin, or hair care, and
a silicone replacement component comprising a recombinant silk polypeptide,
wherein the composition is substantially free of silicone.

2. The composition of claim 1, wherein the silicone replacement component is present in a loading level less than conventional loading levels of a silicone while providing at least the same performance.

3. The composition of claim 2, wherein the performance is one or more of imparting a matte finish; detangling of hair; imparting a soft feel to skin; imparting a silky, smooth, and powdery feel; decrease in glossiness on the skin; enhanced shine on the hair; vibrant and efficient pigment delivery; easy spreadability; quick absorption time; wrinkle blurring effect; hair style retention; heat resistance for hair; UV and pollution defense; and increased viscosity.

4. The composition of any one of the preceding claims, wherein the recombinant silk polypeptide is present in the form of a powder.

5. The composition of claim 4, wherein the silicone replacement composition further comprises a solvent, and the powder is suspended in the solvent.

6. The composition of claim 5, wherein the solvent is an aqueous solvent, a polar solvent, a non-polar solvent, an oil solvent, a wax solvent, or a surfactant.

7. The composition of claim 5 or 6, wherein the silicone replacement composition further comprises a preservative or a chelating agent.

8. The composition of any one of claims 1 to 4, wherein the silicone replacement is the recombinant silk polypeptide.

9. The composition of any one of claims 1 to 4, wherein the silicone replacement comprises the recombinant silk polypeptide suspended in a diluent.

10. The composition of any one of claims 1 to 3, wherein the silicon elastomer replacement comprises the recombinant silk polypeptide as a randomly structured gel.

11. The composition of claim 10, wherein said gel comprises preservative agents or chelating agents.

12. The composition of any one of the preceding claims, wherein 1 part recombinant silk polypeptide is capable of providing a replacement of up to 60 parts silicone.

13. The composition of any one of the preceding claims, wherein the recombinant silk polypeptide is present in an amount of about 0.01 wt % to about 30 wt % based on the total weight of the composition.

14. The composition of claim 13, wherein said composition comprises based on the total weight of the composition 0.01 wt % to 0.5 wt % recombinant silk polypeptide, 0.05 wt % to 5 wt % recombinant silk polypeptide, 0.5 wt % to 5 wt % recombinant silk polypeptide, 1 wt % to 5 wt % recombinant silk polypeptide, or 5 wt % to 30 wt % recombinant silk polypeptide.

15. The composition of any one of the preceding claims, wherein the silicone replacement increases viscosity of the composition as measured by the G′ and G″ rheology curve as compared to a composition having the same amount of silicone, while maintain a G′ and G″ rheology curve having a substantially same shape as a G′ and G″ rheology curve for the composition having the same amount of silicone.

16. The composition of any one of the preceding claims, wherein the silicone replacement comprises the recombinant silk polypeptide suspended in a solvent.

17. The composition of claim 16, wherein the recombinant silk polypeptide is present in an amount of about 1 wt % to about 40 wt % based on the total weight of the silicone replacement.

18. The composition of claim 16 or 17, wherein the solvent is aqueous solvent, an alcohol, or an oil-based solvent.

19. The composition of claim 16 or 17, wherein the solvent is one or more of water, glycerin, deionized water, olive oil, and pentylene glycol.

20. The composition of any one of the preceding claims, wherein the recombinant silk polypeptide is greater than 100 amino acids in length.

21. The composition of any one of the preceding claims, wherein the recombinant silk polypeptide is self-assembled into a semicrystalline state.

22. The composition of claim 21, wherein the crystalline portion of said recombinant silk polypeptide is characterized by beta-sheet crosslinks.

23. The composition of any one of the preceding claims, wherein said recombinant silk has poor solubility in a solvent selected from the group consisting of: water at a pH from 3 to 8, other polar and non polar solvents (for example, hexanol, hexane, benzene), oils, waxes, surfactants (for example, anionic, non-ionic, cationic, amphoteric).

24. The composition of any one of the preceding claims, wherein said recombinant silk forms a heterogeneous dispersion in a solvent selected from the group consisting of: water at a pH from 3 to 8, other polar and nonpolar solvents (hexanol, hexane, benzene), oils, waxes, surfactants (anionic, non-ionic, cationic, amphoteric).

25. The composition of any one of the preceding claims, wherein said recombinant silk polypeptide comprises SEQ ID NO: 1.

26. The composition of any one of the preceding claims, wherein said composition is an SPF formulation, a color cosmetic, wash-off hair shampoo, skin serum, skin primer, or a hair serum.

27. The composition of any one of the preceding claims, wherein said composition comprises water.

28. The composition of any one of claims 1 to 27, wherein said composition comprises water, glycerin, and sodium benzoate.

29. The composition of any one of claims 1 to 27, wherein said composition comprises: Zinc Oxide, Aloe Barbadensis Leaf Juice, Water, Butyrospermum Parkii (Shea) Nut Extract, Camellia Sinesis (Green Tea) Leaf Extract, Caprylic/Capric Triglyceride, Capryloyl Glycerin/Sebacic Acid Copolymer, Caprylyl/Capryl Glucoside, Cetearyl Alcohol, Cetearyl Glucoside, Coco-caprate/caprylate, Coco-Glucoside, Coconut Alkanes, Diheptyl Succinate, Dipotassium Glycyrrhizate, Ethylhexylglycerin, Glycerin, Hydrolyzed Jojoba Esters, Isostearic Acid, Lecithin, Phenoxyethanol, Polyglyceryl-3 Polyricinoleate, Polyhydroxystearic Acid, Potassium Sorbate, Pyrus Malus (Apple) Fruit Extract, Sclerotium Gum, Sodium Benzoate, Sodium Phytate, Squalane, Tocopherol, Xanthan Gum, or any combination thereof.

30. The composition of any one of claims 1 to 27, wherein said composition comprises: water, brassica glycerides, caprylic/capric triglyceride, cetearyl alcohol, CI 77491, cucumber extract, gluconolactone, glycerin, glycerl stearate, Helianthus annuus (sunflower) seed wax, hydrogenated polycyclopentadiene, iron oxides, jojoba ester, lithospermum erythorhzon root extract, plyacrylate crosspolymer-y, polyglycerin-3, polyglyceryl-6 polyricinoleate, silica, Simmondsia chinensis (jojoba) seed oil, sodium benzoate, sodium stearoyl glutamate, tocopherol, or any combination thereof.

31. The composition of any one of claims 1 to 27, wherein said composition comprises: Water, Polyacrylate crosspolymer-6, Glycerin, Citric acid, Gluconolactone, Sodium benzoate, Cetrimonium Chloride, or any combination thereof.

32. The composition of any one of the preceding claims, wherein the composition further comprises a dye.

33. The composition of any one of the preceding claims, wherein the silicone is a silicone elastomer.
